(19) United States
(12) Patent Application Publication
(10) Pub. No.: US 2007/0060586 A1
(43) Pub. Date: Mar. 15, 2007

(54) Nitrooxyderivatives of antihypertensive drugs

(76) Inventors: Piero Del Soldato, Monza (IT); Francesca Benedini, Milano (IT); Ennio Ongini, Segrate (IT)

Correspondence Address: ARENT FOX PLLC, 1050 CONNECTICUT AVENUE, N.W., SUITE 400, WASHINGTON, DC, 20036, US

(21) Appl. No.: 10/581,450

(22) PCT Filed: Dec. 1, 2004
(86) PCT No.: PCT/EP04/13682

§ 371 Date: Oct. 4, 2006

(30) Foreign Application Priority Data
Dec. 2, 2003 (EP) 03104485.2

(51) Int. Cl. A61K 31/5377; A61K 31/4704; A61K 31/405; A61K 31/34; A61K 31/21

(52) U.S. Cl. 514/235.5; 514/469; 514/312; 514/509; 514/415; 558/482; 546/157; 548/503; 544/134

(57) ABSTRACT

The present invention relates to β-adrenergic blockers nitrooxyderivatives of general formula (I): A-(Y—ONO₂)ₛ and enantiomers and diastereoisomers and pharmaceutically acceptable salts thereof, pharmaceutical compositions containing them and their use for the treatment of hypertension, cardiovascular diseases, glaucoma, migraine headache and vascular diseases.

The present invention relates to β-adrenergic blockers derivatives. More particularly, the present invention relates to β-adrenergic blockers nitrooxyderivatives, pharmaceutical compositions containing them and their use for the treatment of hypertension, cardiovascular diseases, glaucoma, migraine headache, vascular diseases and elevated intraocular pressure.

β-adrenergic blockers (β-blockers) are widely used in the treatment of hypertension and cardiovascular diseases Including angina pectoris, arrhythmias, acute myocardial infarction, hypertrophic cardiomyopathy, congestive heart failure. They work to block the effects of catecholamines at receptor sites in the heart, but they differ somewhat in their ability to block receptors in the blood vessels and lungs. Selective β-blockers have their major actions on the heart, some others are weak stimulators of the β-receptor while still blocking the major actions of catecholamines, some block both the β₁ and β₂ receptors in the heart and those in the blood vessels and have no stimulatory activity and some block other cathecolamine receptors that can lead to further vascular effects on blood vessels.

Several side effects are associated with this class of drugs such as muscle fatigue, sleep disturbances, decreased heart rate, hypotension, cold extremities, bronchospasm in asthmatic patients, hypoglycaemia, increased lipids in plasma. Moreover, abrupt withdrawal after long-term treatment with beta-blockers has to be avoided, because an increased sensitivity to β-adrenergic system develops.

U.S. Pat. No. 6,242,432 discloses derivatives of formula A-(X₁—NO₂)ₜₒ having an antithrombotic activity, wherein A is the residue of a β-adrenergic blocker, X₁ is a bivalent connecting bridge and tₒ is 1 or 2. The invention is limited to particular meanings of the bivalent connecting bridge X₁.

U.S. Pat. No. 5,502,237 and U.S. Pat. No. 5,639,904 disclose derivatives of formula R₁—Ar—O—CH₂—CH(OH)—CH₂—NH—CH(CH₃)₂ used for the treatment of cardiovascular affections, wherein R₁ is a chain having at least one nitrooxy group as substituent.

U.S. Pat. No. 4,801,596 discloses aminopropanol derivatives of formula 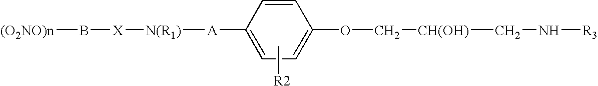
that can be used for prophylaxis and/or treatment of heart and circulatory diseases, wherein R₃ is an alkyl or a nitroxyalkyl radical containing 3 to 8 carbon atoms.

It was an object of the present invention to provide new β-adrenergic blockers nitroxyderivatives having a significantly improved overall pharmacological profile as compared to native β-blockers that are able not only to eliminate or at least reduce the side effects associated with their parent compounds, but also having an improved pharmacological activity and tolerability.

It has been so surprisingly found that the β-adrenergic blockers nitrooxyderivatives of the present invention have a better pharmacological activity and organ protection properties, enhanced effects as anti-inflammatory, and on renal functions. In addition, they are effective in other pathologies including atherosclerosis, diabetes, peripheral vascular diseases (PVD) and elevated intraocular pressure.

In particular, it has been recognized that the β-adrenergic blockers nitrooxyderivatives of the present invention, differently from the above mentioned compounds of the prior art, exhibit an improved activity on the cardiovascular system and enhanced tolerability and can be employed for treating or preventing hypertension, cardiovascular diseases, glaucoma, migraine headache, vascular diseases and elevated intraocular pressure.

Object of the present invention are β-adrenergic blockers nitrooxyderivatives of general formula (I):
A-(Y—ONO₂)ₛ

and enantiomers and diastereoisomers and pharmaceutically acceptable salts thereof, wherein s is an integer equal to 1 or 2, preferably s is 2;

A is selected from the following β-adrenergic blocker residues of formula (II):

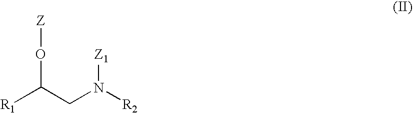

wherein

R₁ is selected from the group consisting of:

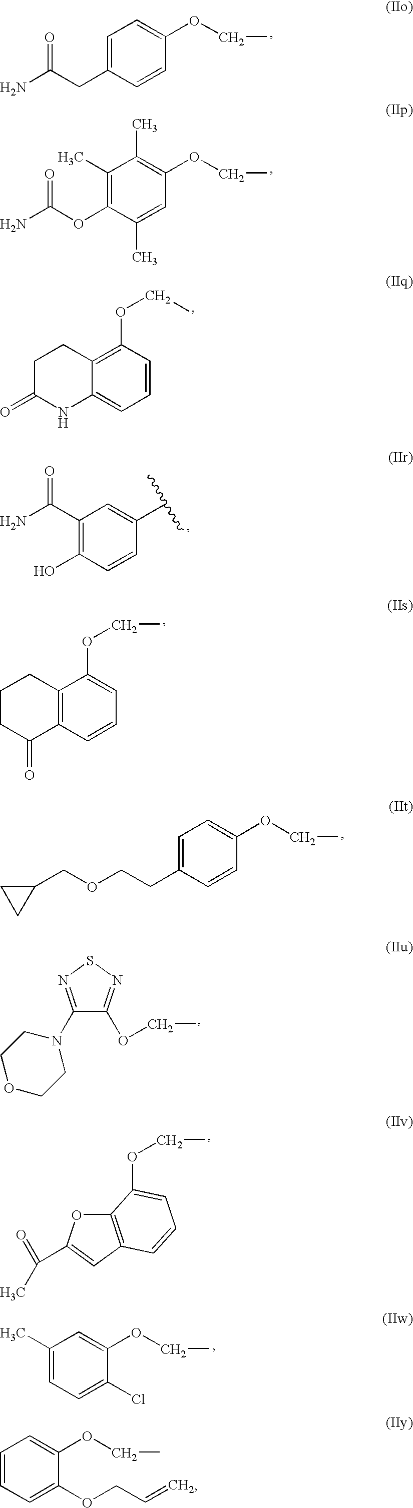

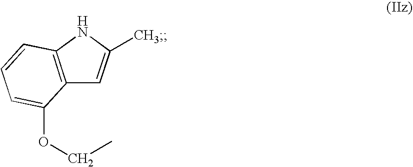

R₂ is selected from the group consisting of: —CH(CH₃)₂, —C(CH₃)₃ or

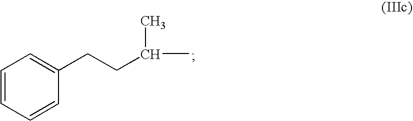

when the radical R₁ has chosen from the formulae (IIo), (IIp), (IIt), (IIu), (IIv), (IIy) or (IIz), R₂ is —CH(CH₃)₂;

when the radical R₁ has chosen from the formulae (IIq), (IIs) or (IIw), R₂ is —C(CH₃)₃;

when the radical R₁ is (IIr), R₂ is (IIIc);

Z is H or is a group capable of binding Y selected from the group consisting of: —C(O)—, —C(O)O— or

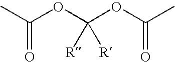

wherein R′ and R″ are the same or different, and are H or straight or branched C₁-C₄ alkyl;

Z₁ is H or a —C(O)— group capable of binding Y;

with the proviso that when s of formula (I) is 1 Z or Z₁ is H;

when s is 2, Z and Z₁ are preferably —C(O)—;

Y is a bivalent radical having the following meaning:

a)

straight or branched C₁-C₂₀ alkylene, preferably C₁-C₁₀, being optionally substituted with one or more of the substituents selected from the group consisting of: halogen atoms, hydroxy, —ONO₂ or T, wherein T is —OC(O)(C₁-C₁₀alkyl)-ONO₂, —O(C₁-C₁₀alkyl)-ONO₂;

b)

cycloalkylene with 5 to 7 carbon atoms into cycloalkylene ring, the ring being optionally substituted with side chains T₁, wherein T₁ is straight or branched alkyl with from 1 to 10 carbon atoms, T₁ is preferably CH₃;

c)

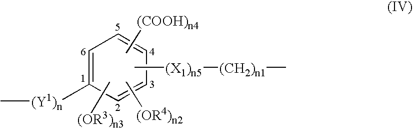

wherein:

n is an integer from 0 to 20, preferably n is an integer from 0 to 10, more preferably n is 0, and n1 is an integer from 1 to 20, preferably from 1 to 10;

n2, n3, n4 and n5 are integers equal or different from each others, equal to 0 or 1;

R³ and R⁴ are independently selected from H or CH₃;

Y¹ is —CH₂— or —(CH₂)ₙₐ—CH═CH— wherein na is an integer from 0 to 20, preferably na is equal to 0;

X₁ is —WC(O)— or —C(O)W—, wherein W is oxygen, sulfur or NH, preferably W is oxygen; with the proviso that:

when s of formula (I) is 1, Z is —(CO)— and in formula (IV) of the bivalent radical Y n2, n3, n4, n5 are equal to 0 then n is 0 and n1 is 1;

when s of formula (I) is 1, Z is —(CO)— and in formula (IV) of the bivalent radical Y n2, n3, n5 are equal to 0, n4 is 1 then n and n1 are different to 1;

d)

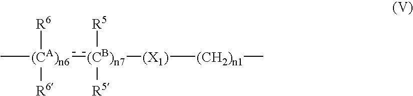

wherein:

n1 is an integer from 1 to 20, preferably from 1 to 10;

X₁ is —WC(O)— or —C(O)W—, wherein W is oxygen, sulfur or NH, preferably W is sulfur;

n6 is an integer from 1 to 20,

n7 is an integer from 0 to 20,

R⁵ and R^{5′} R⁶ and R^{6′} are independently selected from the group consisting of: H, CH₃, OH, NH₂, NHCOCH₃, COOH, CH₂SH and C(CH₃)₂SH; when the bond between the C^{A} and C^{B} carbons is a double bond R⁵ and R⁸ or R^{6′} and R^{5′} are absent;

with the proviso that when Y is selected from the bivalent radicals mentioned under c)-d), the —ONO₂ group is linked to a —(CH₂)ₙ₁— group;

with the proviso that when s of formula (I) is 1 and Z is —(CO)— then the bivalent radical Y has not the meanings under a), b) and d);

e)

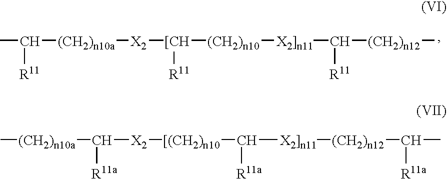

wherein X₂ is O or S,

n10a, n10 and n12 are integer independently selected from 0 to 20,

n10a is preferably selected from 0 to 10,

n10 and n12 are preferably selected from 1 to 10, and

n11 is an integer from 0 to 6, preferably from 0 to 4,

R¹¹ is H, CH₃ or nitrooxy group, preferably R¹¹ is H,

R^{11a} is CH₃ or nitrooxy group;

with the proviso that when in formula (I) s is 1, in formula (II) Z is —(CO)—, in formula (VI) of the bivalent radical Y n10a, n10, n12 are equal to 1 then X can not be an oxygen atom;

f)

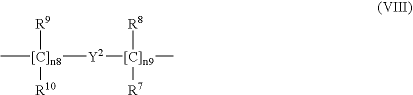

wherein

n8 is an integer from 0 to 10;

n9 is an integer from 1 to 10;

R⁹, R¹⁰, R⁸, R⁷ are same or different, and are H or straight or branched C₁-C₄ alkyl, preferably R⁹, R¹⁰, R⁸, R⁷ are H;

wherein the —ONO₂ group is linked to

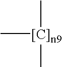

wherein n9 is as defined above;

Y² is an heterocyclic saturated, unsaturated or aromatic 5 or 6 members ring, containing one or more heteroatom/s selected from nitrogen, oxygen, sulfur,

and is selected from the group consisting of

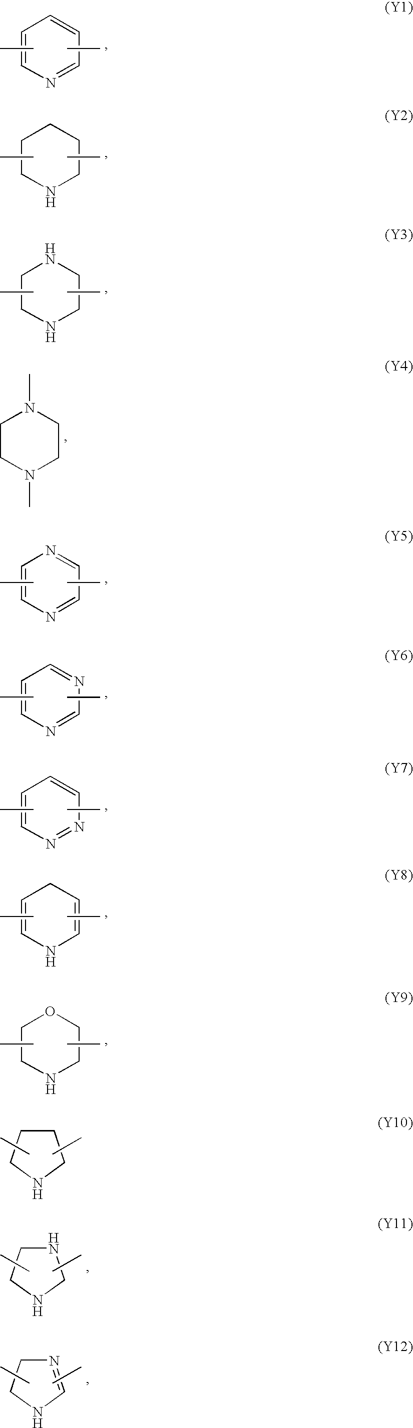

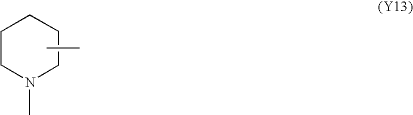

One embodiment of the present invention comprises compounds of formula (I) wherein

s is 2,

A is a β-adrenergic blocker residue of formula (II) as above defined:

Z is a group capable of binding Y selected from the group consisting of:

—C(O)—, —C(O)O— or

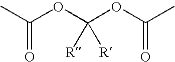

wherein R′ and R″ are the same or different, and are H or straight or branched C₁-C₄ alkyl;

Z₁ is —C(O)—;

preferably Z and Z₁ are —C(O)—;

Y is a bivalent radical having the following meaning:

a)

straight or branched C₁-C₂₀ alkylene, preferably C₁-C₁₀, being optionally substituted with one or more of the substituents selected from the group consisting of: halogen atoms, hydroxy, —ONO₂ or T, wherein T is —OC(O)(C₁-C₁₀alkyl)—ONO₂, —O(C₁-C₁₀alkyl)—ONO₂;

b)

cycloalkylene with 5 to 7 carbon atoms into cycloalkylene ring, the ring being optionally substituted with side chains T₁, wherein T₁ is straight or branched alkyl with from 1 to 10 carbon atoms, T₁ is preferably CH₃;

c)

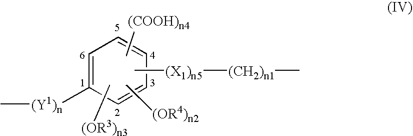

wherein:

n is an integer from 0 to 20, preferably n is an integer from 0 to 10, more preferably n is 0, and n1 is an integer from 1 to 20, preferably from 1 to 10;

n2, n3, n4 and n5 are integers equal or different from each others, equal to 0 or 1;

R³ and R⁴ are independently selected from H or CH₃;

Y¹ is —CH₂— or —(CH₂)ₙₐ—CH═CH— wherein na is an integer from 0 to 20, preferably na is equal to 0;

X₁ is —WC(O)— or —C(O)W—, wherein W is oxygen, sulfur or NH, preferably W is oxygen;

d)

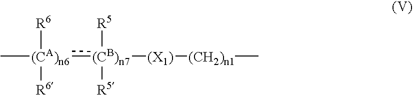

wherein:

n1 is an integer from 1 to 20, preferably from 1 to 10;

X₁ is —WC(O)— or —C(O)W—, wherein W is oxygen, sulfur or NH, preferably W is sulphur;

n6 is an integer from 1 to 20, preferably n6 is 1,

n7 is an integer from 0 to 20, preferably n7 is 1,

R⁵ and R^{5′} R⁶ and R^{6′} are independently selected from the group consisting of: H, CH₃, OH, NH₂, NHCOCH₃, COOH, CH₂SH and C(CH₃)₂SH; when the bond between the C^{A} and C^{B} carbons is a double bond R⁵ and R⁶ or R^{6′} and R^{5′} are absent;

with the proviso that when Y is selected from the bivalent radicals mentioned under c)-d), the —ONO₂ group is linked to a —(CH₂)ₙ₁— group;

e)

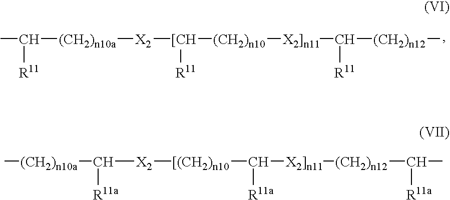

wherein X₂ is O or S,

n10a, n10 and n12 are integer independently selected from 0 to 20,

n10a is preferably selected from 0 to 10,

n10 and n12 are preferably selected from 1 to 10, and

n11 is an integer from 0 to 6, preferably from 0 to 4,

R¹¹ is H, CH₃ or nitrooxy group, preferably R¹¹ is H,

R^{11a} is CH₃ or nitrooxy group;

f)

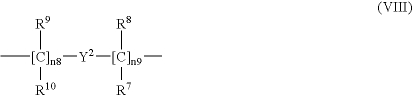

wherein

n8 is an integer from 0 to 10;

n9 is an integer from 1 to 10;

R⁹, R¹⁰, R⁸, R⁷ are same or different, and are H or straight or branched C₁-C₄ alkyl, preferably R⁹, R¹⁰, R⁸, R⁷ are H;

wherein the —ONO₂ group is linked to

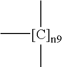

wherein n9 is as defined above;

Y² is an heterocyclic saturated, unsaturated or aromatic 5 or 6 members ring, containing one or more heteroatom is selected from nitrogen, oxygen, sulfur,

and is selected from the group consisting of

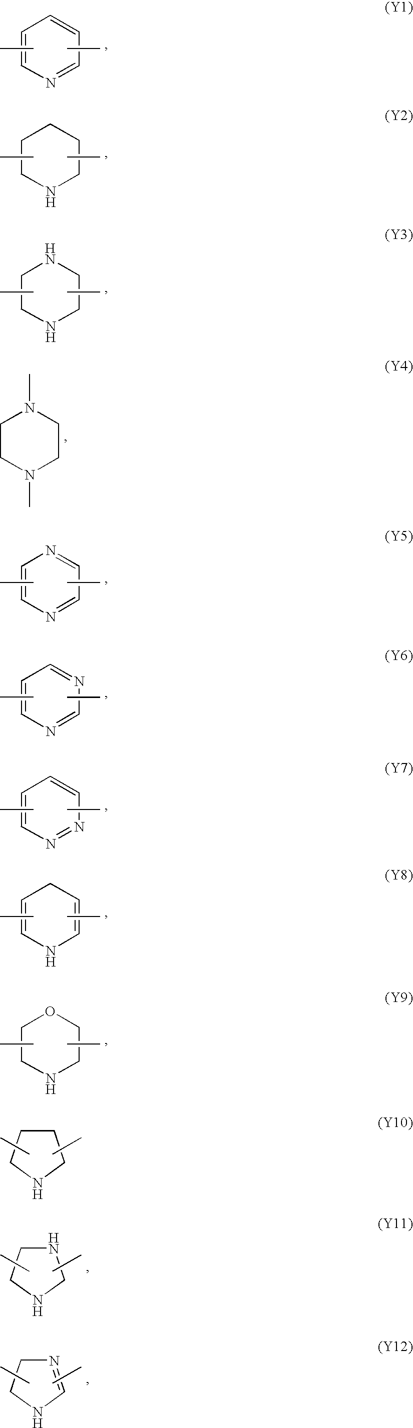

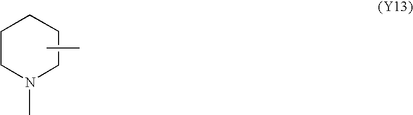

Another embodiment comprises compounds of formula (I) wherein

s is 1,

A is a β-adrenergic blocker residue of formula (II) as above defined:

Z is H,

Z₁ is —(CO)—;

Y is a bivalent radical having the following meaning:

a)

straight or branched C₁-C₂₀ alkylene, preferably C₁-C₁₀, more preferably C₃-C₆ being optionally substituted with one or more of the substituents selected from the group consisting of: halogen atoms, hydroxy, —ONO₂ or T, wherein T is —OC(O)(C₁-C₁₀alkyl)-ONO₂, —O(C₁-C₁₀alkyl)-ONO₂;

b)

cycloalkylene with 5 to 7 carbon atoms into cycloalkylene ring, the ring being optionally substituted with side chains T₁, wherein T₁ is straight or branched alkyl with from 1 to 10 carbon atoms, T₁ is preferably CH₃;

c)

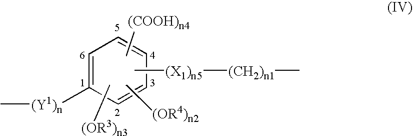

wherein:

n is an integer from 0 to 20, preferably n is an integer from 0 to 10, more preferably n is 0, and n1 is an integer from 1 to 20, preferably from 1 to 10;

n2, n3, n4 and n5 are integers equal or different from each others, equal to 0 or 1;

R³ and R⁴ are independently selected from H or CH₃;

Y¹ is —CH₂— or —(CH₂)ₙₐ—CH═CH— wherein na is an integer from 0 to 20, preferably na is equal to 0;

X₁ is —WC(O)— or —C(O)W—, wherein W is oxygen, sulfur or NH, preferably W is oxygen;

d)

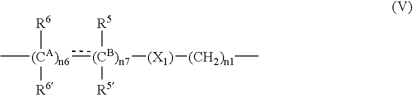

wherein:

n1 is an integer from 1 to 20, preferably from 1 to 10;

X₁ is —WC(O)— or —C(O)W—, wherein W is oxygen, sulfur or NH, preferably W is sulfur;

n6 is an integer from 1 to 20,

n7 is an integer from 0 to 20,

R⁵ and R^{5′} R⁶ and R^{6′} are independently selected from the group consisting of: H, CH₃, OH, NH₂, NHCOCH₃, COOH, CH₂SH and C(CH₃)₂SH; when the bond between the C^{A} and C^{B} carbons is a double bond R⁵ and R⁶ or R^{6′} and R^{5′} are absent;

with the proviso that when Y is selected from the bivalent radicals mentioned under c)-d), the —ONO₂ group is linked to a —(CH₂)ₙ₁— group;

e)

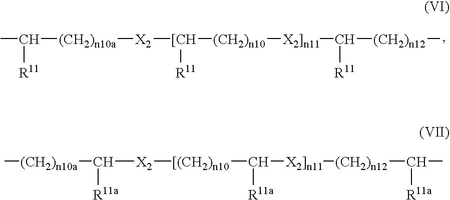

wherein X₂ is O or S,

n10a, n10 and n12 are integer independently selected from 0 to 20,

n10a is preferably selected from 0 to 10,

n10 and n12 are preferably selected from 1 to 10, and

n11 is an integer from 0 to 6, preferably from 0 to 4,

R¹¹ is H, CH₃ or nitrooxy group, preferably R¹¹ is H,

R^{11a} is CH₃ or nitrooxy group;

f)

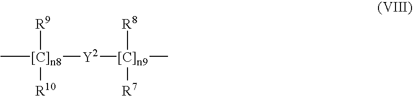

wherein

n8 is an integer from 0 to 10;

n9 is an integer from 1 to 10;

R⁹, R¹⁰, R⁸, R⁷ are same or different, and are H or straight or branched C₁-C₄ alkyl, preferably R⁹, R¹⁰, R⁸, R⁷ are H;

wherein the —ONO₂ group is linked to

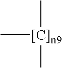

wherein n9 is as defined above;

Y² is an heterocyclic saturated, unsaturated or aromatic 5 or 6 members ring, containing one or more heteroatom/s selected from nitrogen, oxygen, sulfur,

and is selected from the group consisting of

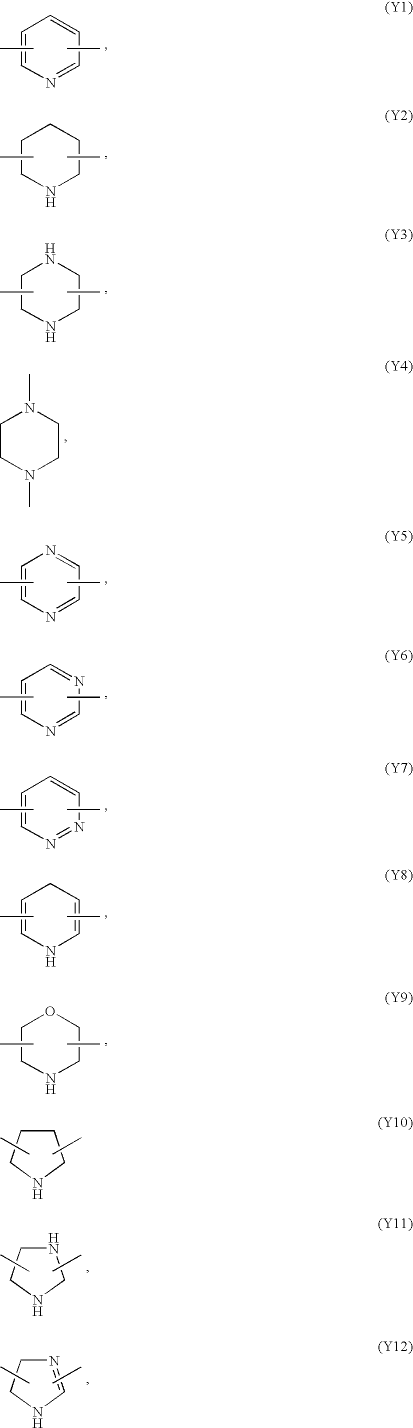

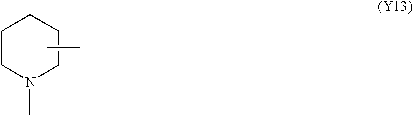

Another embodiment comprises compounds of formula (I) wherein

s is 1,

A is a β-adrenergic blocker residue of formula (II) as above defined:

Z₁ is H,

Z is a group capable of binding Y selected from the group consisting of:

—C(O)—, —C(O)O— or

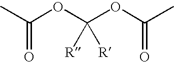

wherein R′ and R″ are the same or different, and are H or straight or branched C₁-C₄ alkyl;

preferably Z is —C(O)—;

Y is a bivalent radical having the following meaning:

c)

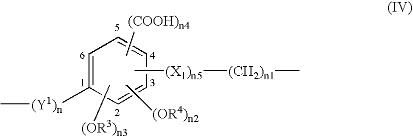

wherein:

n is an integer from 0 to 20, preferably n is an integer from 0 to 10, more preferably n is 0, and n1 is an integer from 1 to 20, preferably from 1 to 10;

n2, n3, n4 and n5 are integers equal or different from each others, equal to 0 or 1;

R³ and R⁴ are independently selected from H or CH₃;

Y¹ is —CH₂— or —(CH₂)ₙₐ—CH═CH— wherein na is an integer from 0 to 20, preferably na is equal to 0;

X₁ is —WC(O)— or —C(O)W—, wherein W is oxygen, sulfur or NH, preferably W is oxygen;

with the proviso that when Z is —C(O)—:

in the bivalent radical Y of formula (IV) n2, n3, n4, n5 are equal to 0 then n is 0 and n1 is 1;

in the bivalent radical Y of formula (IV) n2, n3, n5 are equal to 0, n4 is 1 then n and n1 are different to 1;

e)

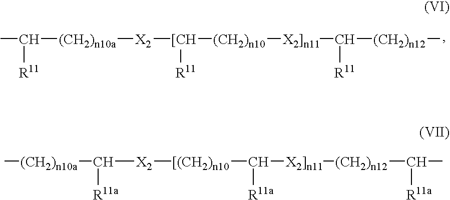

wherein X₂ is O or S,

n10a, n10 and n12 are integer independently selected from 0 to 20,

n10a is preferably selected from 0 to 10,

n10 and n12 are preferably selected from 1 to 10, and

n11 is an integer from 0 to 6, preferably from 0 to 4,

R¹¹ is H, CH₃ or nitrooxy group, preferably R¹¹ is H,

R^{11a} is CH₃ or nitrooxy group;

with the proviso that when Z is —C(O)— and in formula (VI) of the bivalent radical Y n10a,

n10, n12 are equal to 1 then X can not be an oxygen atom;

f)

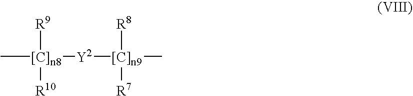

wherein

n8 is an integer from 0 to 10;

n9 is an integer from 1 to 10;

R⁹, R¹⁰, R⁸, R⁷ are same or different, and are H or straight or branched C₁-C₄ alkyl, preferably R⁹, R¹⁰, R⁸, R⁷ are H;

wherein the —ONO₂ group is linked to

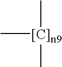

wherein n9 is as defined above;

Y² is an heterocyclic saturated, unsaturated or aromatic 5 or 6 members ring, containing one or more heteroatom/s selected from nitrogen, oxygen, sulfur,

and is selected from the group consisting of

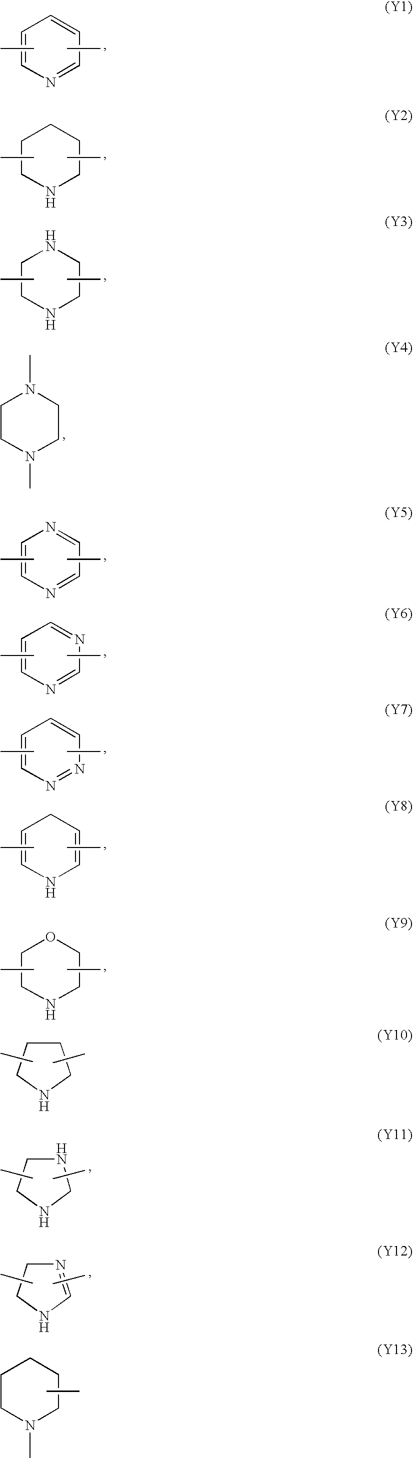

Preferred compounds are those of formula (I) wherein

s is 1

A is a β-adrenergic blocker residue of formula (II) as above defined,

Z is H and Z₁ is —C(O)—,

and the bivalent radical Y have the following meaning:

a) straight C₁-C₁₀ alkylene, preferably C₃-C₆ alkylene;

c)

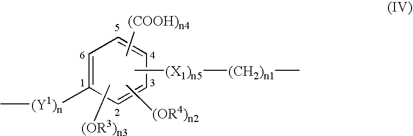

wherein the —ONO₂ group is bound to (CH₂)ₙ₁;

n, n2, n3, n4, n5 are equal to 0,

n1 is 1 and the —(CH₂)ₙ₁— group is bound to the phenyl ring through the [C]₂ or the [C]₃ or the [C]₄; or

n, n2, n5 are 1,

n3 and n4 are equal to 0, and

n1 is an integer from 1 to 10,

Y¹ is —(CH₂)ₙₐ—CH═CH— wherein na is 0,

X₁ is —WC(O)— wherein W is oxygen and the WC(O) group is bound to the phenyl ring through the [C]₄,

R⁴ is CH₃ and the (OR⁴) group is bound to the phenyl ring through the [C]₃;

d)

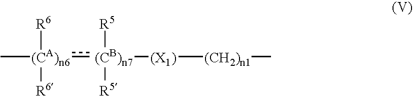

wherein

the —ONO₂ is bound to the —(CH₂)ₙ₁— group;

n1 is an integer from 1 to 10, n6 and n7 are 1, X₁ is —WC(O)— wherein W is sulfur,

R⁵, R^{5′} and R^{6′} are H,

R⁶ is NHCOCH₃.

Another group of preferred compounds are those of formula (I) wherein s is 1,

A is a β-adrenergic blocker residue of formula (II) as above defined,

Z₁ is H and Z is —C(O)—, and

the bivalent radical Y have the following meaning:

c)

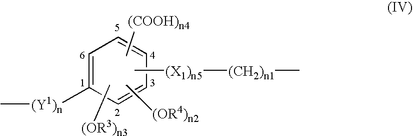

wherein the —ONO₂ group is bound to (CH₂)ₙ₁;

n, n2, n3, n4, n5 are equal to 0,

n1 is 1 and the —(CH₂)ₙ₁— group is bound to the phenyl ring through the [C]₂ or the [C]₃ or the [C]₄; or

n, n2, n5 are 1,

n3 and n4 are equal to 0, and

n1 is an integer from 1 to 10,

Y¹ is —(CH₂)ₙₐ—CH═CH— wherein na is 0,

X₁ is —WC(O)— wherein W is oxygen and the WC(O) group is bound to the phenyl ring through the [C]₄,

R⁴ is CH₃ and the (OR⁴) group is bound to the phenyl ring through the [C]₃;

d)

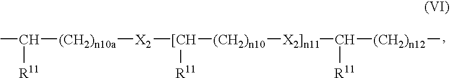

wherein

X₂ is O or S, and n10a and n11 are 0, n12 is 1 and R¹¹ is H and the —ONO₂ group is bound to (CH₂)ₙ₁₂.

Another group of preferred compounds are those of formula (I) wherein s is 2,

A is a β-adrenergic blocker residue of formula (II) as above defined,

Z₁ and Z are —C(O)—, and

the bivalent radical Y have the following meaning:

a) straight C₁-C₁₀ alkylene, preferably C₃-C₆ alkylene;

c)

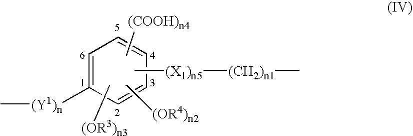

wherein the —ONO₂ group is bound to (CH₂)ₙ₁;

n, n2, n3, n4, n5 are equal to 0,

n1 is 1 and the —(CH₂)ₙ₁— group is bound to the phenyl ring through the [C]₂ or the [C]₃ or the [C]₄;

or n, n2, n5 are 1,

n3 and n4 are equal to 0, and

n1 is an integer from 1 to 10,

Y¹ is —(CH₂)ₙₐ—CH═CH— wherein na is 0,

X₁ is —WC(O)— wherein W is oxygen and the WC(O) group is bound to the phenyl ring through the [C]₄,

R⁴ is CH₃ and the (OR⁴) group is bound to the phenyl ring through the [C]₃;

d)

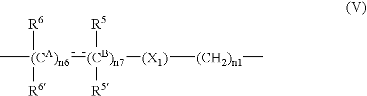

wherein

the —ONO₂ is bound to the —(CH₂)ₙ₁— group;

n1 is an integer from 1 to 10,

n6 and n7 are 1,

X₁ is —WC(O)— wherein W is sulfur,

R⁵, R^{5′} and R^{6′} are H, R⁶ is NHCOCH₃.

Preferred compounds of formula (I) according to the present invention are the following:

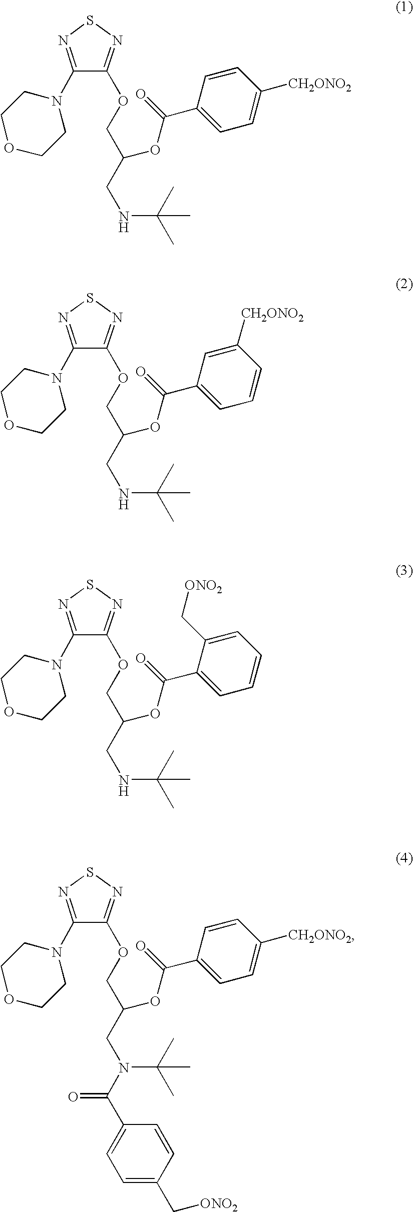

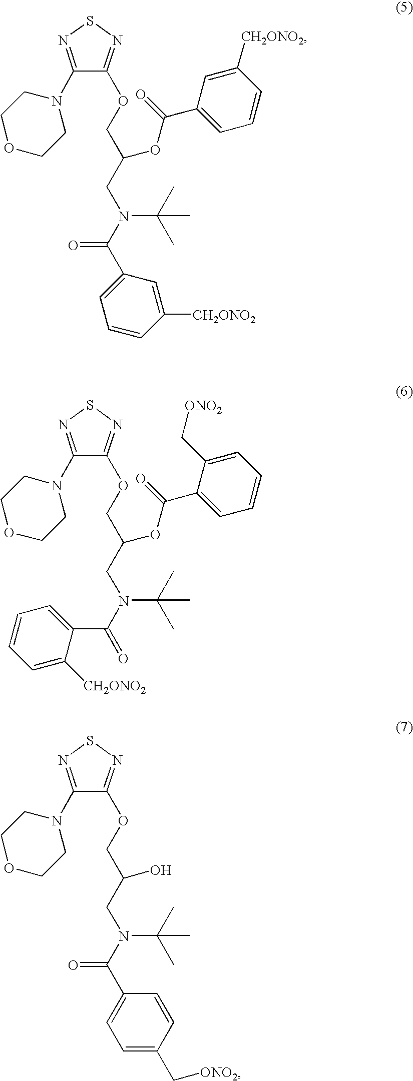

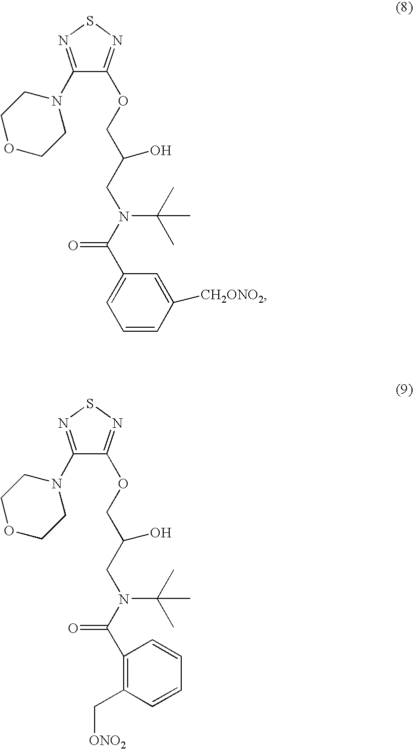

Examples of “straight or branched C₁-C₂₀ alkylene” include, but are not limited to, methylene, ethylene, propylene, isopropylene, n-butylene, pentylene, n-hexylene and the like.

As stated above, the invention includes also the pharmaceutically acceptable salts of the compounds of formula (I) and stereoisomers thereof.

Examples of pharmaceutically acceptable salts are either those with inorganic bases, such as sodium, potassium, calcium and aluminium hydroxides, or with organic bases, such as lysine, arginine, triethylamine, dibenzylamine, piperidine and other acceptable organic amines.

The compounds according to the present invention, when they contain in the molecule one salifiable nitrogen atom, can be transformed into the corresponding salts by reaction in an organic solvent such as acetonitrile, tetrahydrofuran with the corresponding organic or inorganic acids.

Examples of pharmaceutical acceptable organic acids are: oxalic, tartaric, maleic, succinic, citric acids. Examples of pharmaceutical acceptable inorganic acids are: nitric, hydrochloric, sulphuric, phosphoric acids. Salts with nitric acid are preferred.

The compounds of the invention which have one or more asymmetric carbon atoms can exist as optically pure enantiomers, pure diastereomers, enantiomers mixtures, diastereomers mixtures, enantiomer racemic mixtures, racemates or racemate mixtures. Within the object of the invention are also all the possible isomers, stereoisomers and their mixtures of the compounds of formula (I).

The compounds and compositions of the present invention can be administered by any available and effective delivery system including but not limited to, orally, bucally, parenterally, by inhalation spray, by topicall application, by injection, transdermally, or rectally (e.g. by the use of suppositories) in dosage unit formulations containing conventional nontoxic pharmaceutically acceptable carriers, adjuvants, and vehicles. Parenteral includes subcutaneous injections, intravenous, intramuscular, intrasternal injection, or infusion technique.

Solid dosage forms for oral administration can include for example capsule, tablets, pills, powders, granules and gel. In such solid dosage forms, the active compounds can be admixed with at least one inert diluent such as sucrose, lactose or starch. Such dosage form can also comprise, as normal practice, additional substance other than inert diluent, e.g., lubricating agent such as magnesium stearate.

Injectable preparations, for example sterile injectable aqueous or oleaginous suspensions can be formulated according to the known art using suitable dispersing agents, wetting agents and/or suspending agents.

The composition of this invention can further include conventional excipients, i.e., pharmaceutical acceptable organic or inorganic substances which do not deleteriously react with the active compounds.

The doses of β-adrenergic blockers nitrooxyderivatives can be determinated by standard clinique technique and are in the same ranges or less than as described for commercially available compounds as reported in the: Physician's Desk Reference, Medical Economics Company, Inc., Oradell, N.J., 58^{th} Ed., 2004; The pharmacological basis of therapeutics, Goodman and Gilman, J. G. Hardman, L. e. Limbird, 20^{th} Ed.

Experimental: Synthesis Procedure

The compounds of the invention can be synthesized as shown in Schemes 1 to 6. The compounds of general formula (I) A-(Y—ONO₂)ₛ, defined in Schemes 1-3 as compounds of formula D, wherein s is 1, Y is as above defined and A is a β-adrenergic blocker residue of formula (II), wherein Z is —C(O)— and Z₁ is H, the enantiomers, diastereoisomer and a pharmaceutically acceptable salt thereof, can be prepared as outlined in Schemes 1 -3.

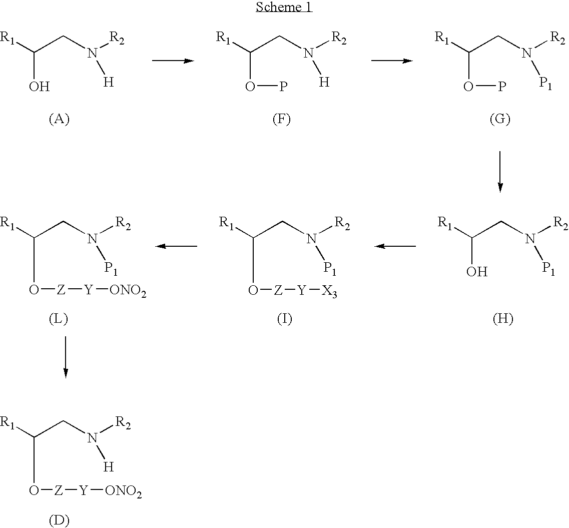

Compounds of formula (i) wherein R₁, R₂, Z and Y are as above defined, P₁ is an amine protecting group such as tert-butyloxycarbonyl ester (t-Boc) and X₃ is an halogen atom preferably Cl, Br and I, are converted to compounds of formula (L) wherein R₁, R₂, P₁, Z and Y are as above defined, by reaction with AgNO₃ in a suitable organic solvent such as acetonitrile, tetrahydrofurane, a silver nitrate molar excess is preferably used and the reaction is carried out, in the dark, at a temperature from room temperature to the boiling temperature of the solvent. The compounds of formula (L) are converted to the compounds of formula (D) by deprotecting the amine group (strong acid, such as HCl in dioxane or trifluoroacetic acid, is used to remove a t-butyl carbamate). Other preferred methods for removing the amine protecting groups are those described in T. W. Greene “Protective groups in organic synthesis”, Harvard University Press, 1980.

The compounds of formula (H) wherein R₁, R₂, Z, P₁ and Y are as above defined, are converted to the esters of formula (i) wherein R₁, R₂, Y, Z, X₃ and P₁ are as above defined, by reaction with an appropriate acid (Q1) of formula X₃—Y—COOH wherein Y and X₃ are as above defined. The reaction is generally carried out in an inert organic solvent such as N,N′-dimethylformamide, tetrahydrofuran, benzene, toluene, dioxane, a polyhalogenated aliphatic hydrocarbon at a temperature from 0° C. to 50° C. In presence of a dehydrating agent such as dycyclohexylcarbodiimide DCC or 1-ethyl-3-(3-dimethylaminopropyl)carbodiimide hydrochloride (EDAC HCl) with a catalyst, such as 4-N,N-dimethylaminopyridine (DMAP).

The compounds of formula (H) wherein R₁, R₂ and P₁ are as above defined, can be obtained by deprotecting the hydroxylic group of the compounds of formula (G) wherein R₁, R₂ are as above defined and P is a hydroxylic protecting group such as silyl ethers, such as trimethylsilyl or tert-butyl-dimethylsilyl and those described in T. W. Greene “Protective groups in organic synthesis”, Harvard University Press, 1980. Fluoride ion is the preferred method for removing silyl ether protecting group.

The compounds of formula (G) wherein R₁, R₂, P and P₁ are as above defined, can be obtained by reacting the compounds of formula (F) wherein R₁, R₂ and P are as above defined with a suitable amine protecting group (P₁) as above described.

The alcohol group of the compounds of formula (A) wherein R₁, R₂ are as above defined, is protected to afford the compounds of formula (F) wherein R₁, R₂ are as above defined Preferred protecting groups for the alcohol moiety are silyl ethers, such as trimethylsilyl or tert-butyl-dimethylsilyl.

The compounds (A) wherein R₁, R₂ are as above defined are commercially available, the acids of formula X₃—Y—COOH wherein X₃ is as above defined, are commercially available.

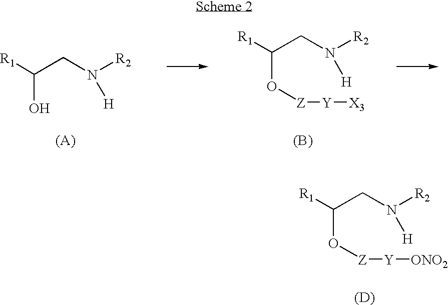

Compounds of formula (B) wherein R₁, R₂, Z, Y are as above defined and X₃ is an halogen atom, such as Cl, Br and I, are converted to compounds of formula (D) wherein R₁, R₂, Z and Y are as above defined, by reaction with AgNO₃ in a suitable organic solvent such as acetonitrile, tetrahydrofurane, a silver nitrate molar excess is preferably used and the reaction is carried out, in the dark, at a temperature from room temperature and the boiling temperature of the solvent.

The compounds of formula (B) wherein R₁, R₂, Z, Y and X₃ are as above defined can be obtained by reaction of compounds of formula (A) with an appropriate acid chloride (Q) of formula X₃—Y—C(O)Cl, wherein X₃ is chosen among chlorine, bromine, and Y is as above defined. The reaction of formation of the ester is carried out in an inert organic solvent such as N,N′-dimethylformamide, tetrahydrofuran, benzene, toluene, chloroform in presence of a base as triethylamine, pyridine at a temperature from room temperature and 50° C. The reaction is completed within a time range from 30 minutes to 24 hours. Alternatively the compounds of formula (B) can be obtained by reaction of a compound of formula (A) with an acid (Q1) of formula X₃—Y—C(O)OH in the presence of a dehydrating agent as dicyclohexylcarbodiimide (DCC) or N′-(3-dimethylaminopropyl)-N-ethylcarbodiimide hydrochloride (EDAC) and a catalyst, such as N,N-dimethylamino pyridine. The reaction is carried out in an inert organic solvent such as N,N′-dimethylformamide, tetrahydrofuran, benzene, toluene, dioxane, a polyhalogenated aliphatic hydrocarbon at a temperature from 0° C. and 50° C. The reaction is completed within a time range from 30 minutes to 36 hours.

The compounds of formula (Q1), where X₃ is an halogen atom are commercially available or can be obtained from the corresponding commercially available hydroxyl acid by well known reactions, for example by reaction with thionyl or oxalyl chloride, halides of P^{III} or P^{V} in solvents inert such as toluene, chloroform, DMF, etc.

The compounds (A) wherein R₁, R₂ are as above defined are commercially available.

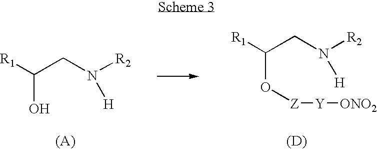

Alternatively the compounds of formula (D) can be obtained as described below. The compounds of formula A are converted to the ester (D) by reaction of the alcohol group with a nitrooxyderivative, containing activated acylating group, of formula Cl(O)C—Y—ONO₂. The nitrooxy compounds can be obtained from the corresponding alcohols of formula Cl(O)C—Y—OH by reaction with nitric acid and acetic anhydride in a temperature range from −50° C. to 0° C. or from the corresponding halogen derivatives of formula Cl(O)C—Y-Hal by reaction with silver nitrate in the presence of an inert solvent such as acetonitrile, tetrahydrofurane. A silver nitrate molar excess is preferably used and the reaction is carried out, in the dark, a temperature from the boiling temperature and room temperature. The reaction is completed within a time range from 30 minutes to 3 days.

The compounds of general formula (I) A-(Y—ONO₂)ₛ, defined in Scheme 4 as compounds of formula (D1), wherein s is 1, Y is as above defined and A is a β-adrenergic blocker residue of formula (II), wherein Z is —C(O)O— and Z₁ is H, the enantiomers, diastereoisomer and a pharmaceutically acceptable salt thereof, can be prepared as outlined in Scheme 4.

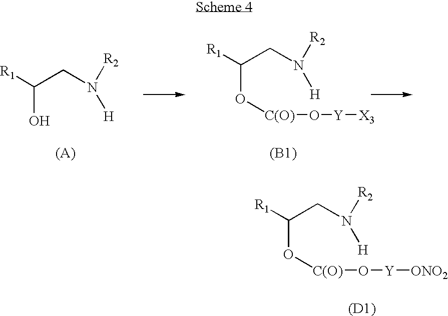

The compounds of formula (B1) wherein R₁, R₂, Y are as above defined and X₃ is an halogen atom, such as Cl, Br and I, are converted to compounds of formula (D1) wherein R₁, R₂, and Y are as above defined, by reaction with AgNO₃ in a suitable organic solvent such as acetonitrile, tetrahydrofurane, a silver nitrate molar excess is preferably used and the reaction is carried out, in the dark, at a temperature from room temperature and the boiling temperature of the solvent.

The compounds of formula (A) wherein R₁ and R₂ are as above defined are converted to the compounds (B1) by reaction with an appropriate compound (Q2) having formula X₃—Y—OC(O)Cl wherein X₃ is Cl, Br or I, and Y is as defined above. The reaction is generally carried out in presence of a base in an aprotic polar or non-polar solvent such as THF or CH₂Cl₂ at temperatures range between 0°-65° C. or in a double phase system H₂O/Et₂O at temperatures range between 20°-40° C.

The compounds of formula (Q2) are commercially available or can be obtained from the corresponding alcohols by reaction with triphosgene in presence of an organic base.

The compounds of general formula (I) A-(Y—ONO₂)ₛ, defined in Scheme 5 as compounds of formula (D), wherein s is 1, Y is as above defined and A is a β-adrenergic blocker residue of formula (II), wherein Z is 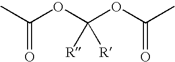
wherein R′ and R″ are as above defined and Z₁ is H, the enantiomers, diastereoisomer and a pharmaceutically acceptable salts thereof, may be prepared as outlined in Scheme 5:

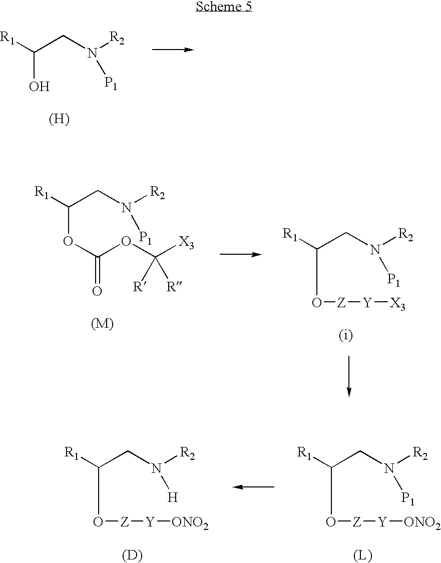

The compounds of formula (I) wherein R₁, R₂, Z and Y are as above defined, P₁ is an amine protecting group such as tert-butyloxycarbonyl ester (t-Boc) and X₃ is an halogen atom such as Cl, Br and I, are converted to compounds of formula (L) wherein R₁, R₂, P₁, Z and Y are as above defined, by reaction with AgNO₃ in a suitable organic solvent such as acetonitrile, tetrahydrofurane, a silver nitrate molar excess is preferably used and the reaction is carried out, in the dark, at a temperature from room temperature and the boiling temperature of the solvent. The compounds of formula (L) are converted to the compounds of formula (D) by deprotecting the amine group (strong acid, such as HCl in dioxane or trifluoroacetic acid, is used to remove a t-butyl carbamate). Other preferred methods for removing the amine protecting groups are those described in T. W. Greene “Protective groups in organic synthesis”, Harvard University Press, 1980.

The compounds of formula (i) wherein R₁, R₂, Y, X₃, Z and P₁ are as above defined, can be obtained by reacting the compounds of formula (M) wherein R₁, R₂, P₁, R′, R″ and X₃ are as above defined, with an acid (Q1) of formula X₃—Y—COOH wherein X₃ is an halogen atom and Y is as above defined. The reaction is carried out in an inert organic solvent such as N,N′-dimethylformamide, tetrahydrofuran, benzene, toluene, dioxane, a polyhalogenated aliphatic hydrocarbon at a temperature from 0° C. and 50° C. in the presence of a dehydrating agent such as dycyclohexylcarbodiimide DCC or 1-ethyl-3-(3-dimethylaminopropyl)carbodiimide hydrochloride (EDAC HCl) with a catalyst, such as 4-N,N-dimethylaminopyridine (DMAP).

The reaction is complete within a time range from 30 minutes to 24 hours.

The compounds of formula (M) wherein R₁, R₂, P₁, R′, R″ and X₃ are as above defined, can be obtained by reacting compounds the of formula (H) with an acyl compound (S) of formula X₃—C(R′)(R″)—OC(O)X₃ wherein X₃ is an halogen atom. The reaction is carried out in presence of an organic or inorganic base in a polar solvent as DMF, THF, acetonitrile at a temperature in the range from −5° C. to 60° C. or in a double phase system according to methods well known in the literature.

The amine group of the compounds (A) is protected to afford the compounds of formula (H) wherein P₁ is a suitable amine protecting group such as tert-butyloxycarbonyl ester (t-Boc) The compounds (S) are commercially available.

The compounds of general formula (I) A-(Y—ONO₂)ₛ, defined in Scheme 6 as compounds of formula (E), wherein s is 2, Y is as above defined and A is a β-adrenergic blocker residue of formula (II), wherein Z₁ and Z are —C(O)—, the enantiomers, diastereoisomer and a pharmaceutically acceptable salt thereof, can be synthesized as shown in Scheme 6.

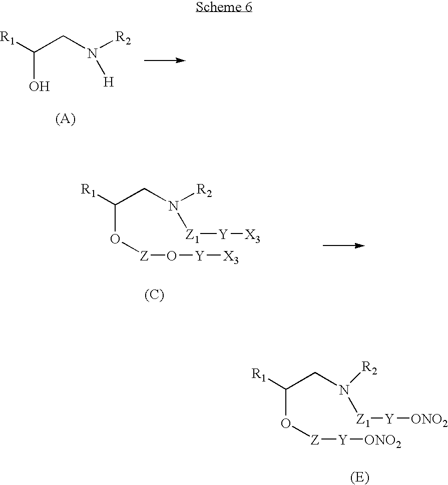

Compound of formula (C) wherein R₁, R₂, Z, Z₁ and Y are as above defined and X₃ is an halogen atom, such as Cl, Br and I, are converted to compounds of formula (E) wherein R₁, R₂, Z and Y are as above defined, by reaction with AgNO₃ in a suitable organic solvent such as acetonitrile, tetrahydrofurane, a silver nitrate molar excess is preferably used and the reaction is carried out, in the dark, at a temperature from room temperature and the boiling temperature of the solvent.

The compounds of formula (C) wherein R₁, R₂, Z, Z₁, Y and X₃ are as above defined can be obtained by reaction of compounds of formula (A) with an appropriate acid chloride (Q) of formula X₃—Y—C(O)Cl, wherein X₃ is chosen among chlorine, bromine, and Y is as above defined. The reaction is carried out in an inert organic solvent such as N,N′-dimethylformamide, tetrahydrofuran, benzene, toluene, chloroform in presence of a base as triethylamine, pyridine at a temperature from room temperature and 50° C. The reaction is completed within a time range from 30 minutes to 24 hours.

Alternatively the compounds of formula (C) can be obtained by reaction of compounds of formula (A) with an acid (Q1) of formula X₃—Y—COOH in the presence of a dehydrating agent such as dicyclohexylcarbodiimide (DCC) or N′-(3-dimethylaminopropyl)-N-ethylcarbodiimide hydrochloride (EDAC) and a catalytic amount of N,N-dimethylamino pyridine. The reaction is carried out in an inert organic solvent such as as N,N′-dimethylformamide, tetrahydrofuran, benzene, toluene, dioxane, a polyhalogenated aliphatic hydrocarbon at a temperature from 0° C. and 50° C. The reaction is completed within a time range from 30 minutes to 36 hours.

The compounds of formula (Q1), where X₃ is an halogen atom are commercially available or can be obtained from the corresponding commercially available hydroxyl acids by well known reactions, for example by reaction with thionyl or oxalyl chloride, halides of P^{III} or P^{V} in solvents inert such as toluene, chloroform, DMF, etc.

The compounds (A) wherein R₁, R₂ are as above defined are commercially available. The compounds of formula (D) can also be obtained as described below. The compounds of formula A are converted to the compounds (E) by reaction with a nitrooxy derivative of formula Cl(O)C—Y—ONO₂ containing an activated acylating group.

The nitrooxy-compounds can be obtained from the corresponding alcohols of formula Cl(O)C—Y—OH by reaction with nitric acid and acetic anhydride in a temperature range from −50° C. to 0° C. or from the corresponding halogen derivatives of formula Cl(O)C—Y-Hal by reaction with silver nitrate In the presence of an inert solvent such as acetonitrile, tetrahydrofurane. A silver nitrate molar excess is preferably used and the reaction is carried out, in the dark, a temperature from the boiling temperature and room temperature. The reaction is completed within a time range from 30 minutes to 3 days.

### EXAMPLES

The following non-limiting examples further describe and enable of ordinary skilled in the art to make and use the present invention.

### Example 1

### 4-(Nitrooxymethyl)benzoic acid (S)-1-[(1,1-dimethylethyl)amino]-3-[[4-(4-morpholinyl)-1,2,5thiadiazol-3-yl]oxy]-2-propanoate maleate salt

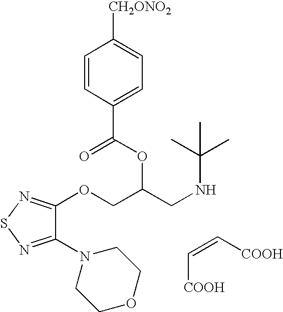

### 1a. 4-(Chloromethyl)benzoic acid (S)-1-[(1,1-dimethylethyl)amino]-3-[[4-(4-morpholinyl)-1,2,5-thiadiazol-3-yl]oxy]-2-propanoate

To a solution of timolol (3.5 g, 11 mmol) in chloroform (200 ml) 4-chloromethyl benzoic acid (1.9 g, 11 mmol), EDAC (3.16 g, 16.5 mmol) and N,N-dimethylaminopyridine (catalytic amount) were added. The reaction was stirred for 12 hours at room temperature. The solution was washed with water, dried over sodium sulphate and concentrated under reduced pressure. The residue was purified by flash chromatography eluting with chloroform/isopropanol 10/0.5 to give the title compound 3 g as a white powder.

### 1b. 4-(Nitrooxymethyl)benzoic acid (S)-1-[(1,1-dimethylethyl)amino]-3-[[4-(4-morpholinyl)-1,2,5-thiadiazol-3-yl]oxy]-2-propanoate

A solution of the product of example 1a (1 g, 2.1 mmol) and silver nitrate (0.71 g, 4.21 mmol) in acetonitrile (50 ml) was stirred at 60° C., in the dark, for 36 hours. The precipitated (silver salts) was removed by filtration and the solvent was evaporated under vacuum. The residue was treated with chloroform and water. The organic layer was dried over sodium sulfate and the solvent was evaporeted. The residue was purified by flash chromatography, eluting with chloroform/isopropanol 10/0.5 to give the title compound 0.6 g as white powder.

### 1c. 4-(Nitrooxymethyl)benzoic acid (S)-1-[(1,1-dimethylethyl)amino]-3-[[4-(4-morpholinyl)-1,2,5-thiadiazol-3-yl]oxy]-2-propanoate maleate salt

To a solution of the product of the example 1b (0.6 g, 1.2 mmol) in acetone (100 ml) maleic acid (0.14 g, 1.2 mmol) was added. The reaction was stirred at room temperature for 1 hours. The precipitated was filtered, washed with acetone and dried under vacuum to afford the title compound 0.6 g as a white powder.

M.p.=160° C.

¹H-NMR (CDCl₃) δ (ppm): 7.99 (2H,d); 7.42 (2H,d); 5.93 (2H,s); 5.87 (1H,m); 5.46 (2H, s); 4.82 (1H,dd); 4.71 (1H,dd); 3.73 (4H,m); 3.44 (4H,m);1.49 (9H,s).

### Example 2

### 4-(Nitrooxymethyl)benzoic acid (S)-1-[(1,1-dimethylethyl)[(4-nitrooxymethyl)benzoyl]amino]-3-[[4-(4-morpholinyl)-1,2,5-thiadiazol-3-yl]oxy]-2-propanoate

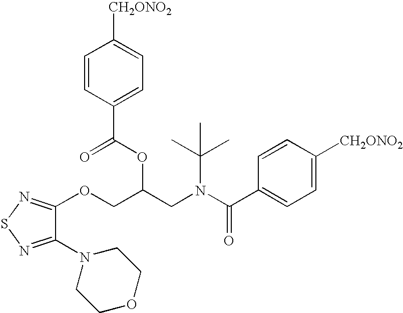

### 2a. 4-(Chloromethyl)benzoic acid (S)-1-[(1,1-dimethylethyl)[(4-chloromethyl)benzoyl]amino]-3-[[4-(4-morpholinyl)-1,2,5-thiadiazol-3-yl]oxy]-2-propanoate

To a solution of timolol hydrocloride (8 g, 22.66 mmol) in chloroform (130 ml) a mixture of 4-chloromethyl benzoylchloride (4.28 g, 22.66 mmol) and triethylamine (6.2 ml, 44.66 mmol) in chloroform (70 ml) was added dropwise. The reaction was stirred for 24 hours at room temperature. The solution was treated with water and diethyl ether, the organic layers were dried over sodium sulfate and concentrated under reduced pressure. The residue was purified by flash chromatography, eluting with chloroform/isopropanol 10/0.3 to give the title compound 3 g as powder.

### 2b. 4(Nitrooxymethyl)benzoic acid (S)-1-[(1,1-dimethylethyl)[(4-nitrooxymethyl)benzoyl]amino]-3-[[4-(4-morpholinyl)-1,2,5-thiadiazol-3-yl]oxy]-2-propanoate

A solution of the product of example 2a (1.5 g, 2.4 mmol) and silver nitrate (1.23 g, 7.2 mmol) in acetonitrile (100 ml) was stirred at 60° C., in the dark, for 36 hours. The precipitated (silver salts) was removed by filtration and the filtrate was concentrated. The residue was treated with chloroform and water and the organic layer was dried over sodium sulfate and concentrated under reduced pressure. The residue was purified by flash chromatography eluting with chloroform/isopropanol 10/0.2 to give the title product 0.95 g as a yellow powder.

M.p.=44-46° C.

¹H-NMR (CDCl₃) δ (ppm): 7.95 (2H,d); 7.50 (2H,d); 7.38(4H,s); 5.79 (1H,m); 5.75(2H,s), 5.74 (2H,s); 4.50 (1H,dd); 4.30 (1H,dd); 3.95 (1H,dd); 3.85 (1H,dd); 3.59 (4H,m); 3.34 (4H,m); 1.60 (9H,s).

### Example 3

### (S)-1-[(1,1-dimethylethyl)[(4-nitrooxymethyl)benzoyl]amino]-3-[[4-(4-morpholinyl)-1,2,5-thiadiazol-3-yl]oxy]-2-propanol

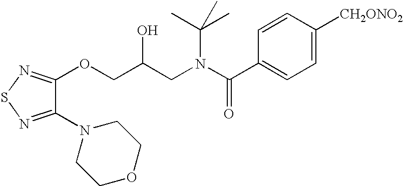

### 3a. (S)-1-[(1,1-dimethylethyl)amino]-3-[[4-(4-morpholinyl)-1,2,5-thiadiazol-3-yl]oxy]-2-propan-1-tert-butyidimethylsilylether

To a solution of timolol (2 g, 6.32 mmol) in N,N-dimethylformamide (10 ml) tert-butyldimethylsilylchloride (1.15 g, 7.58 mmol) and imidazole (1 g, 15.8 mmol) were added. The reaction was stirred for 2 hours at room temperature. The solution was concentrated under reduced pressure and the residue was purified by flash chromatography eluting with chloroform/isopropanol 10/0.3 to give the title compound 1.5 g.

### 3b. (S)-1-[(1,1-dimethylethyl)[(4-chloromethyl)benzoyl]amino]-3-[[4-(4-morpholinyl)-1,2,5-thiadiazol-3-yl]oxy]-2-propan-1- tert-butyldimethylsilylether

To a solution of the product of the example 3a (0.7 g, 1.62 mmol) in chloroform (50 ml) 4-chloromethyl benzoylchloride (0.46 g, 2.44 mmol) and triethylamine (0.39 ml, 2.44 mmol) were added. The reaction was stirred for 24 hours at room temperature. The solution was treated with water and diethyl ether, the organic layers were dried over sodium sulphate and concentrated under reduced pressure. The residue was purified by flash chromatography eluting with n-hexane/ethyl acetate 7/3 to give the title product (0.7 g).

### 3c. (S)-1-[(1,1-dimethylethyl)[(4-chloromethyl)benzoyl]amino]-3-[[4-(4-morpholinyl)-1,2,5-thiadiazol-3-yl]oxy]-2-propanol

To a solution of the product of example 3b (0.6 g, 1.03 mmol) in tetrahydrofuran (50 ml) cooled at 0° C., a solution of tetrabutylamonium floride in tetrahydrofuran 1M (0.54 ml, 2.05 mmol) was added. The reaction was stirred for 30 minutes at room temperature. The solution was concentrated under reduced pressure and the residue was purified by flash chromatography eluting with n-hexane/ethyl acetate 1/1 to give the title product 0.2 g.

### 3d. (S)-1-[(1,1-dimethylethyl)[(4-nitrooxymethyl)benzoyl]amino]-3-[[4-(4-morpholinyl)-1,2,5-thiadiazol-3-yl]oxy]-2-propanol

A solution of the product of example 3c (0.15 g, 0.32 mmol) and silver nitrate (0.11 g, 0.64 mmol) in acetonitrile (50 ml) was stirred at 65° C., in the dark, for 32 hours. The precipitated (silver salts) was removed by filtration and the filtrate concentrate. The residue was treated with methylene chloride and water and the organic layer was dried over sodium sulfate and concentrated under reduced pressure. The residue was purified by flash chromatography eluting with n-hexane/ethyl acetate 45/65 to afford the title compound 0.65 g as a white powder.

M.p.=50-54° C.

¹H-NMR (CDCl₃) δ (ppm): 7.40 (4H,s); 5.44 (2H,s); 4.33-4.18(3H,m), 3.79 (4H,dd); 3.64-3.50 ((2H,m); 4.46 (4H,dd); 3.00 (1H,s); 1.53 (9H,s).

### Example 4

Measurements of CGMP in Rat PC12 Cell Line.

cGMP contributes to the function and interaction of several vascular cell types and its dysfunction is involved in major cardiovascular diseases such as hypertension, diabetic complications, atherosclerosis, and tissue infarction. Therefore the extent of cGMP formation elicited by the compounds of the inventions was evaluated in the rat pheochromocytoma (PC12) cell line.

Tested Compounds

1) Timolol (parent compound)
2) 4-(Nitrooxymethyl)benzoic acid (S)-1-[(1,1-dimethylethyl)[(4-nitrooxymethyl)benzoyl]amino]-3-[[4-(4-morpholinyl)-1,2,5-thiadiazol-3-yl]oxy]-2-propanoate (compound of example 1)
3) 4-(Nitrooxymethyl)benzoic acid (S)-1-[(1,1-dimethylethyl)[(4-nitrooxymethyl)benzoyl]amino]-3-[[4-(4-morpholinyl)-1,2,5-thiadiazol-3-yl]oxy]-2-propanoate (compound of example 2)
4) (S)-1-[(1,1-dimethylethyl)[(4-nitroaxymethyl)benzoyl]amino]-3-[[4-(4-morpholinyl)-1,2,5-thiadiazol-3-yl]oxy]-2-propanol (compound of example 3)
   Method

Cells were maintained at 37° C. in DMEM medium enriched with 10% horse serum and 5% foetal bovine serum under 5% CO₂ atmosphere. At the time of experiments the cells were washed once with Hank's Balanced Salt Solution (HBSS) supplemented with 0.05% ascorbic acid and preincubated in the same buffer for 10 min in a floating water bath. After the preincubation step, cells were exposed for additional 45 min to either control conditions or increasing concentrations of test compounds ranging from 0.1 to 25 μM, in the presence of the phosphodiesterase inhibitor, IBMX (100 μM) and the NO-independent activator of soluble guanylyl cyclase, YC-1 (20 μM). The reaction was terminated by the removal of the incubating buffer and consecutive addition of 100 μl of absolute ethanol. The organic extracts were then evaporated to dryness and the residues dissolved in aqueous buffer for quantitative determination of intracellular cGMP levels using the cGMP enzyme immunoassay kit.

The obtained results reported in Table 1 are expressed as EC₅₀ (μM) and efficacy Emax (% of vehicle). As shown in the table the nitroderivatives of timolol elicited consistent increase of intracellular cGMP formation in PC12 cell line. Conversely, this effect was not shared by the parent compound.

TABLE 1
Effects of nitroxyderivatives of timolol and ann of timolol
on cGMP formation in PC12 cells

| Compound | EC₅₀ (μM) | Eₘₐₓ(% of vehicle) |
|---|---|---|
| Timolol | Not effective | Not effective |
| Compound of example 2 | 1.3 | 480 |
| Compound of example 1 | 12.6 | 796 |
| Compound of example 3 | 18.5 | 866 |

1. A compound of general formula (I) A-(Y—ONO₂)ₛ and the enantiomers, diastereoisomers and pharmaceutically acceptable salts thereof, wherein s is an integer equal to 1 or 2;
A is selected from the following β-adrenergic blockers residues of formula (II): 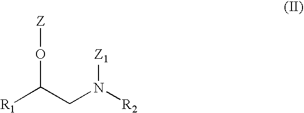
wherein
R₁ is selected from the group consisting of: 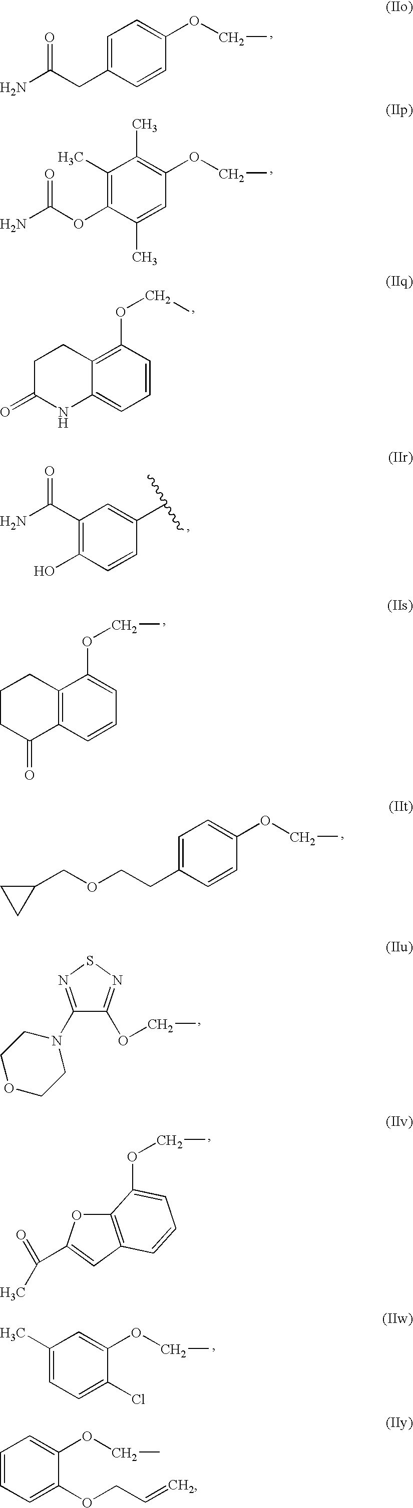 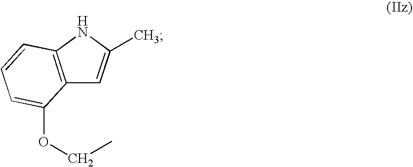
R₂ is selected from the group consisting of: —CH(CH₃)₂, —C(CH₃)₃ or 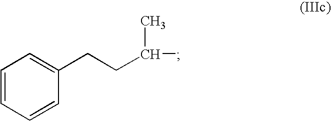
when the radical R₁ has chosen from the formulae (IIo), (IIp), (IIt), (IIu), (IIv), (IIy) or (IIz), R₂ is —CH(CH₃)₂;
when the radical R₁ has chosen from the formulae (IIq), (IIs) or (IIw), R₂ is —C(CH₃)₃;
when the radical R₁ is (IIr), R₂ is (IIIc);
Z is H or is a group capable of binding Y selected from the group consisting of: —C(O)—, —C(O)O— or 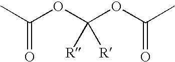
wherein R′ and R″ are the same or different, and are H or straight or branched C₁-C₄ alkyl;
Z₁ is H or a —C(O)-group capable of binding Y;
with the proviso that when s of formula (I) is 1 Z or Z₁ is H;
Y is a bivalent radical having the following meaning:
a)
straight or branched C₁-C₂₀ alkylene being optionally substituted with one or more of the substituents selected from the group consisting of halogen atoms, hydroxy, —ONO₂ or T, wherein T is —OC(O)(C₁-C₁₀alkyl)—ONO₂, —O(C₁-C₁₀alkyl)—ONO₂;
b)
cycloalkylene with 5 to 7 carbon atoms into cycloalkylene ring, the ring being optionally substituted with side chains T₁, wherein T₁ is straight or branched alkyl with from 1 to 10 carbon atoms;
c) 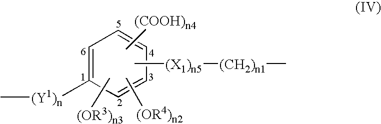
wherein:
n is an integer from 0 to 20, and n1 is an integer from 1 to 20;
n2, n3, n4 and n5 are integers equal or different from each other, equal to 0 or 1;
R³ and R⁴ are independently selected from H or CH₃;
Y¹ is —CH₂— or —(CH₂)ₙₐ—CH═CH— wherein na is an integer from 0 to 20;
X₁ is —WC(O)— or —C(O)W—, wherein W is oxygen, sulfur or NH;
with the proviso that:
when s of formula (I) is 1, Z is —(CO)— and in formula (IV) of the bivalent radical Y n2, n3, n4, n5 are equal to 0 then n is 0 and n1 is 1;
when s of formula (I) is 1, Z is —(CO)— and in formula (IV) of the bivalent radical Y n2, n3, n5 are equal to 0, n4 is 1 then n and n1 are different to 1;
d) 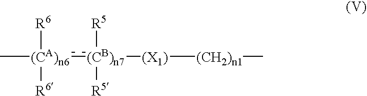
wherein:
n1 is an integer from 1 to 20;
X₁ is —WC(O)— or —C(O)W—, wherein W is oxygen, sulfur or NH;
n6 is an integer from 1 to 20,
n7 is an integer from 0 to 20,
R⁵ and R^{5′} R⁶ and R^{6′} are independently selected from the group consisting of H, CH3, OH, NH₂, NHCOCH₃, COOH, CH₂SH and C(CH₃)₂SH; when the bond between the C^{A} and C^{B} carbons is a double bond R⁵ and R⁶ or R^{6′} and R^{5′} are absent;
with the proviso that when Y is selected from the bivalent radicals mentioned under c)-d), the —ONO₂ group is linked to a —(CH₂)ₙ₁— group;
with the proviso that when s of formula (I) is 1 and Z is —(CO)— then the bivalent radical Y has not the meanings under a), b) and d);
e) 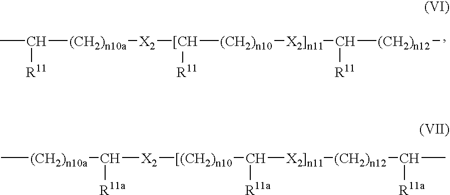
wherein X₂ is O or S,
n10a, n10 and n12 are integer independently selected from 0 to 20,
n11 is an integer from 0 to 6,
R¹¹ is H, CH₃ or nitrooxy group;
R^{11a} is CH₃ or a nitrooxy group;
with the proviso that when in formula (I) s is 1, in formula (II) Z is —(CO)—, in formula (VI) of the bivalent radical Y n10a, n10, n12 are equal to 1 then X can not be an oxygen atom;
f) 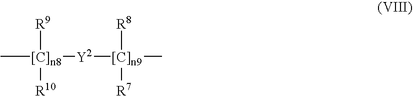
wherein:
n8 is an integer from 0 to 10;
n9 is an integer from 1 to 10;
R⁹, R¹⁰, R⁸, R^{7′} are the same or different, and are H or straight or branched C₁-C₄ alkyl;
wherein the —ONO₂ group is linked to 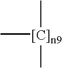
wherein n9 is as defined above;
Y² is an heterocyclic saturated, unsaturated or aromatic 5 or 6 members ring, containing one or more heteroatoms selected from nitrogen, oxygen, sulfur, and is selected from the group consisting of:

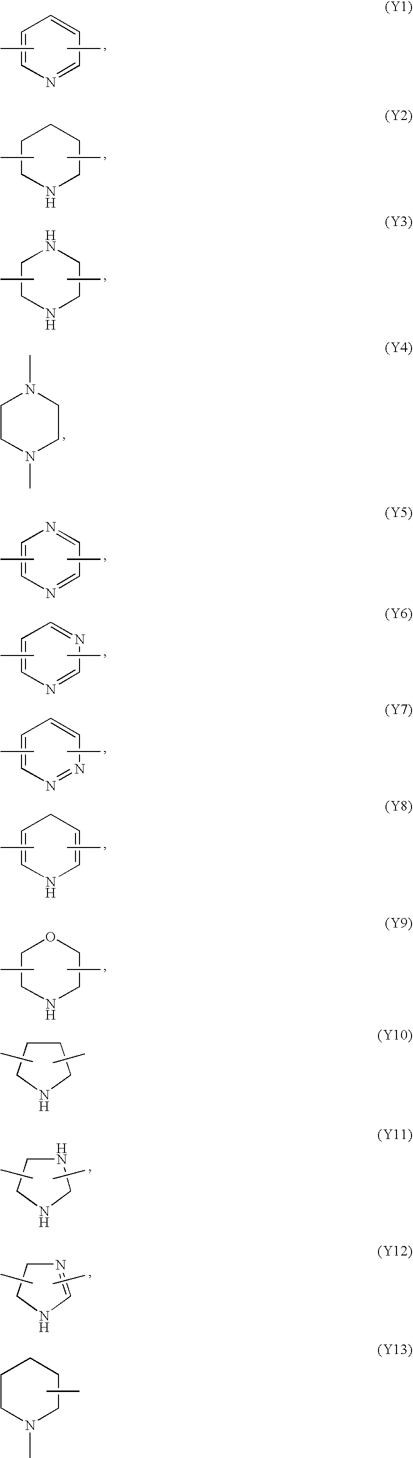

2. A compound and the enantiomers, diastereoisomers and pharmaceutically acceptable salts thereof according to claim 1 wherein s is equal to 1 and Z₁ is H.

3. A compound and the enantiomers, diastereoisomers and pharmaceutically acceptable salts thereof according to claim 2 wherein Z is —C(O)—.

4. A compound and enantiomers and diastereoisomers and pharmaceutically acceptable salts thereof according to claim 3 wherein
Y is 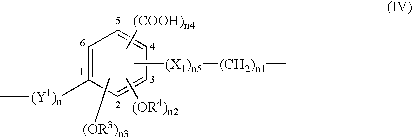
wherein
n, n2, n3, n4 and n5 are equal to 0
n1 is an integer equal to 1;

5. A compound and the enantiomers, diastereoisomers and pharmaceutically acceptable salts thereof according to claim 3 wherein
Y is 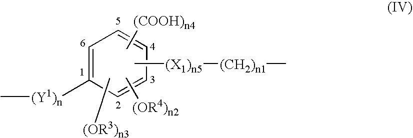
wherein
n, n2, n5 are 1,
n3 and n4 are equal to 0,
n1 is an integer from 1 to 10,
Y¹ is —(CH₂)ₙₐ—CH═CH— wherein na is 0,
X₁ is —WC(O)— wherein W is oxygen and X₁ is bound to the phenyl ring through the [C]₄,
R⁴ is CH₃ and the (OR⁴) group is bound to the phenyl ring through the [C]₃.

6. A compound and the enantiomers, diastereoisomers and pharmaceutically acceptable salts thereof according to claim 3 wherein
Y is 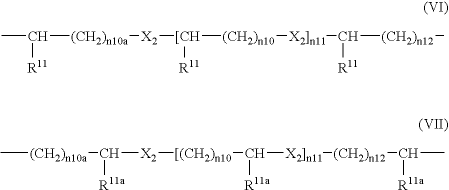
wherein
X₂ is O or S,
n10a, n10 and n12 are integers independently selected from 2 to 20;
n11 is an integer from 0 to 6;
R¹¹ is H, CH₃ or a nitrooxy group;
R^{11a} is CH₃ or a nitrooxy group.

7. A compound and the enantiomers, diastereoisomers and pharmaceutically acceptable salts thereof according to claim 2 wherein Z is —C(O)O—.

8. A compound and the enantiomers, diastereoisomers and pharmaceutically acceptable salts thereof according to claim 7 wherein
Y is a straight or branched C₁-C₂₀ alkylene being optionally substituted with one or more of the substituents selected from the group consisting of halogen atoms, hydroxy, —ONO₂ or T, wherein T is —OC(O)(C₁-C₁₀alkyl)—ONO₂, —O(C₁-C₁₀alkyl)—ONO₂.

9. A compound and the enantiomers, diastereoisomers and pharmaceutically acceptable salts thereof according to claim 8 wherein Y is a straight or branched C₁-C₁₀ alkylene.

10. A compound and the enantiomers, diastereoisomers and pharmaceutically acceptable salts thereof according to claim 7 wherein Y is 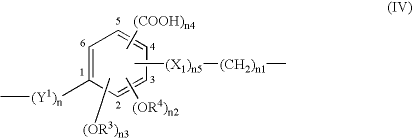
wherein
n is an integer from 0 to 20,
n1 is an integer from 1 to 20;
n2, n3, n4 and n5 are integers equal or different from each other, equal to 0 or 1;
R³ and R⁴ are independently selected from H or CH₃;
Y¹ is —CH₂— or —(CH₂)ₙₐ—CH═CH— wherein na is an integer from 0 to 20;
X₁ is —WC(O)— or —C(O)W—, wherein W is oxygen, sulfur or NH.

11. A compound and the enantiomers, diastereoisomers and pharmaceutically acceptable salts thereof according to claim 10 wherein
n2, n3, n4, n5 are equal to 0,
n1 is 1,
n is an integer from 0 to 10,
Y¹ is CH₂.

12. A compound and the enantiomers, diastereoisomers and pharmaceutically acceptable salts thereof according to claim 7 wherein Y is 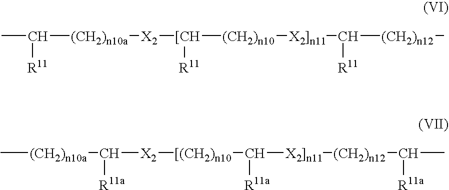
wherein X₂ is O or S,
n10a, n10 and n12 are integers independently selected from 0 to 20;
n11 is an integer from 0 to 6;
R¹¹ is H, CH₃ or a nitrooxy group;
R^{11a} is CH₃ or a nitrooxy group.

13. A compound and the enantiomers, diastereoisomers and pharmaceutically acceptable salts thereof according to claim 12 wherein Y is 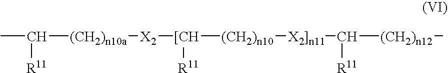
wherein
X₂ is O or S,
n10a and n11 are 0,
n12 is 1, and
R¹¹ is H;
wherein the —ONO₂ group is bound to the —(CH₂)ₙ₁₂— group.

14. A compound and the enantiomers, diastereoisomers and pharmaceutically acceptable salts thereof according to claim 7 wherein Y is 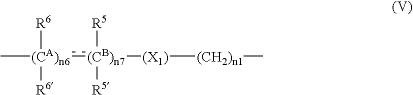
wherein:
n1 is an integer from 1 to 20;
X₁ is —WC(O)— or a —C(O)W—, wherein W is oxygen, sulfur or NH.
n6 is an integer from 1 to 20,
n7 is an integer from 0 to 20,
R⁵ and R^{5′} R⁶ and R^{6′} are independently selected from the group consisting of: H, CH₃, OH, NH₂, NHCOCH₃, COOH, CH₂SH and C(CH₃)₂SH; when the bond between the C^{A} and C^{B} carbons is a double bond R⁵ and R⁶ or R^{6′} and R^{5′} are absent.

15. A compound and the enantiomers, diastereoisomers and pharmaceutically acceptable salts thereof according to claim 2 wherein Z is

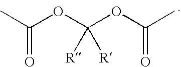

16. A compound and the enantiomers, diastereoisomers and pharmaceutically acceptable salts thereof according to claim 15 wherein Y is a straight or branched C₁-C₂₀ alkylene being optionally substituted with one or more of the substituents selected from the group consisting of halogen atoms, hydroxy, —ONO₂ or T, wherein T is —OC(O)(C₁-C₁₀alkyl)—ONO₂, —O(C₁-C₁₀alkyl)—ONO₂.

17. A compound and the enantiomers, diastereoisomers and pharmaceutically acceptable salts thereof according to claim 16 wherein Y is a straight or branched C₁-C₁₀ alkylene.

18. A compound and the enantiomers, diastereoisomers and pharmaceutically acceptable salts thereof according to claim 15 wherein Y is 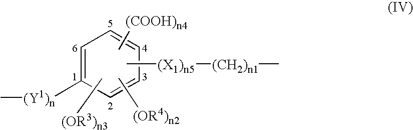
wherein
n is an integer from 0 to 20,
n1 is an integer from 1 to 20,
n2, n3, n4 and n5 are integers equal or different from each other, equal to 0 or 1;
R³ and R⁴ are independently selected from H or CH₃;
Y¹ is —CH₂— or —(CH₂)ₙₐ—CH═CH— wherein na is an integer from 0 to 20;
X₁ is —WC(O)— or —C(O)W—, wherein W is oxygen, sulfur or NH.

19. A compound and the enantiomers, diastereoisomers and pharmaceutically acceptable salts thereof according to claim 18 wherein
n2, n3, n4, n5 are equal to 0,
n1 is 1,
n is an integer from 0 to 10,
Y¹ is CH₂.

20. A compound and the enantiomers, diastereoisomers and pharmaceutically acceptable salts thereof according to claim 1 wherein Z and Z₁ are —C(O)—.

21. A compound and the enantiomers, diastereoisomers and pharmaceutically acceptable salts thereof according to claim 20 wherein Y is a straight or branched C₁-C₂₀ alkylene being optionally substituted with one or more of the substituents selected from the group consisting of halogen atoms, hydroxy, —ONO₂ or T, wherein T is —OC(O)(C₁-C₁₀alkyl)—ONO₂, —O(C₁-C₁₀alkyl)—ONO₂.

22. A compound and the enantiomers, diastereoisomers and pharmaceutically acceptable salts thereof according to claim 21 wherein Y is a straight or branched C₁-C₁₀ alkylene.

23. A compound and the enantiomers, diastereoisomers and pharmaceutically acceptable salts thereof according to claim 20 wherein Y is 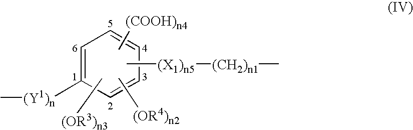
wherein
n is an integer from 0 to 20,
n1 is an integer from 1 to 20,
n2, n3, n4 and n5 are integers equal or different from each other, equal to 0 or 1,
R³ and R⁴ are independently selected from H or CH3;
Y¹ is —CH₂— or —(CH₂)ₙₐ—CH═CH— wherein na is an integer from 0 to 20;
X₁ is —WC(O)— or —C(O)W—, wherein W is oxygen, sulfur or NH.

24. A compound and the enantiomers, diastereoisomers and pharmaceutically acceptable salts thereof according to claim 23 wherein
n2, n3, n4, n5 are equal to 0,
n1 is 1,
n is an integer from 0 to 10,
Y¹ is CH₂.

25. A compound and the enantiomers, diastereoisomers and pharmaceutically acceptable salts thereof according to claim 23 wherein
n, n2, n5 are 1,
n3 and n4 are equal to 0,
n1 is an integer from 1 to 10,
Y¹ is —(CH₂)ₙₐ—CH═CH— wherein na is 0,
X₁ is —WC(O)— wherein W is oxygen and X₁ is bound to the phenyl ring through the [C]₄,
R⁴ is CH₃ and the group (OR⁴) is bound to the phenyl ring through the [C]₃.

26. A compound and the enantiomers, diastereoisomers and pharmaceutically acceptable salts thereof according to claim 20 wherein Y is 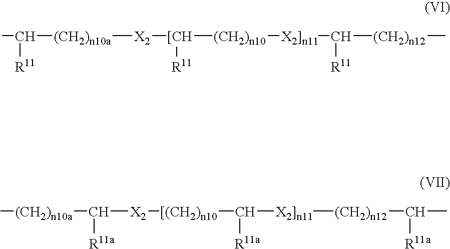
wherein
X₂ is O or S,
n10a, n10 and n12 are integers independently selected from 0 to 20;
n11 is an integer from 0 to 6;
R¹¹ is H, CH₃ or a nitrooxy group;
R^{11a} is CH₃ or a nitrooxy group.

27. A compound and the enantiomers, diastereoisomers and pharmaceutically acceptable salts thereof according to claim 26 wherein Y is 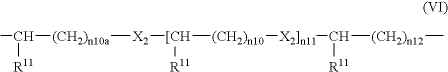
wherein
X₂ is O or S,
n10a and n11 are 0,
n12 is 1,
R¹¹ is H;
wherein the —ONO₂ group is bound to the —(CH2)ₙ₁₂— group.

28. A compound and the enantiomers, diastereoisomers and pharmaceutically acceptable salts thereof according to claim 20 wherein Y is 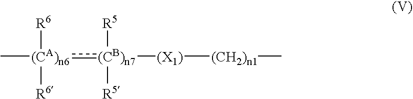
wherein:
n1 is an integer from 1 to 20;
X₁ is —WC(O)— or a —C(O)W—, wherein W is oxygen, sulfur or NH.
n6 is an integer from 1 to 20,
n7 is an integer from 0 to 20,
R⁵ and R^{5′} R⁶ and R^{6′} are independently selected from the group consisting of: H, CH₃, OH, NH₂, NHCOCH₃, COOH, CH₂SH and C(CH₃)₂SH;
when the bond between the C^{A} and C^{B} carbons is a double bond R⁵ and R⁶ or R^{6′} and R^{5′} are absent.

29. A compound and the enantiomers, diastereoisomers and pharmaceutically acceptable salts thereof according to claim 28 wherein
n1 is an integer from 1 to 10,
n6 and n7 are 1;
X₁ is —WC(O)— wherein W is sulfur,
R⁵, R^{5′} and R^{6′} are H,
R⁶ is NHCOCH₃,
with the proviso that the —ONO₂ group is bound to the —(CH₂)ₙ₁— group.

30. A compound and the enantiomers, diastereoisomers and pharmaceutically acceptable salts thereof according to claim 1 wherein s is 1, Z is H and Z₁ are —C(O)—.

31. A compound and the enantiomers, diastereoisomers and pharmaceutically acceptable salts thereof according to claim 30 wherein Y is a straight or branched C₁-C₂₀ alkylene being optionally substituted with one or more of the substituents selected from the group consisting of halogen atoms, hydroxy, —ONO₂ or T, wherein T is —OC(O)(C₁-C₁₀alkyl)—ONO₂, —O(C₁-C₁₀alkyl)—ONO₂.

32. A compound and the enantiomers, diastereoisomers and pharmaceutically acceptable salts thereof according to claim 31 wherein Y is a straight or branched C₁-C₁₀ alkylene.

33. A compound and the enantiomers, diastereoisomers and pharmaceutically acceptable salts thereof according to claim 30 wherein Y is 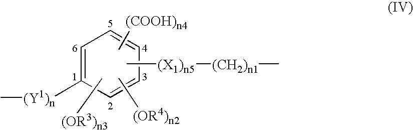
wherein
n is an integer from 0 to 20,
n1 is an integer from 1 to 20;
n2, n3, n4 and n5 are integers equal or different from each other, equal to 0 or 1;
R³ and R⁴ are independently selected from H or CH₃;
Y¹ is —CH₂— or —(CH₂)ₙₐ—CH═CH— wherein na is an integer from 0 to 20;
X₁ is —WC(O)— or —C(O)W—, wherein W is oxygen, sulfur or NH.

34. A compound and the enantiomers, diastereoisomers and pharmaceutically acceptable salts thereof according to claim 33 wherein
n2, n3, n4, n5 are equal to 0,
n1 is 1,
n is an integer from 0 to 10
Y¹ is CH₂.

35. A compound and the enantiomers, diastereoisomers and pharmaceutically acceptable salts thereof according to claim 33 wherein
n, n2, n5 are 1,
n3 and n4 are equal to 0,
n1 is an integer from 1 to 10,
Y¹ is —(CH₂)ₙₐ—CH═CH— wherein na is 0,
X₁ is —WC(O)—, wherein W is oxygen and X₁ is bound to the phenyl ring through the [C]₄,
R⁴ is CH₃ and the group (OR⁴) is bound to the phenyl ring through the [C]₃.

36. A compound and the enantiomers, diastereoisomers and pharmaceutically acceptable salts thereof according to claim 30 wherein Y is 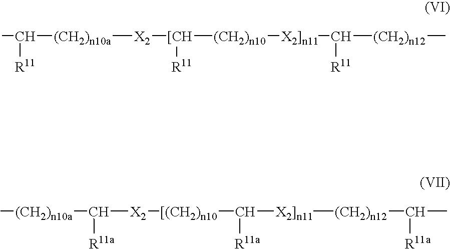
wherein
X₂ is O or S,
n10a, n10 and n12 are integers independently selected from 0 to 20;
n11 is an integer from 0 to 6;
R¹¹ is H, CH₃ or a nitrooxy group;
R^{11a} is CH₃ or a nitrooxy group.

37. A compound and the enantiomers, diastereoisomers and pharmaceutically acceptable salts thereof according to claim 36 wherein Y is 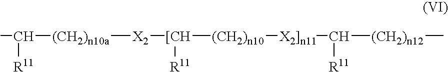
wherein X₂ is O or S,
n10a and n11 are 0,
n12 is 1,
R¹¹ is H,
wherein the —ONO₂ group is bound to the —(CH₂)ₙ₁₂— group.

38. A compound and the enantiomers, diastereoisomers and pharmaceutically acceptable salts thereof according to claim 30 wherein Y is 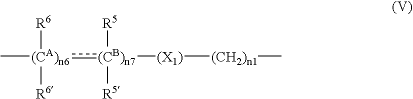
wherein:
n1 is an integer from 1 to 20;
X₁ is —WC(O)— or a —C(O)W—, wherein W is oxygen, sulfur or NH.
n6 is an integer from 1 to 20,
n7 is an integer from 0 to 20,
R⁵ and R^{5′} R⁶ and R^{6′} are independently selected from the group consisting of: H, CH₃, OH, NH₂, NHCOCH₃, COOH, CH₂SH and C(CH₃)₂SH;
when the bond between the C^{A} and C^{B} carbons is a double bond R⁵ and R⁶ or R^{6′} and R^{5′} are absent.

39. A compound and the enantiomers, diastereoisomers and pharmaceutically acceptable salts thereof according to claim 38 wherein
n1 is an integer from 1 to 10,
n6 and n7 are 1;
X₁ is WC(O)— wherein W is sulfur;
R⁵, R^{5′} and R_{6′} are H, R⁶ is NHCOCH₃;
with the proviso that the —ONO₂ group is bound to the —(CH₂)ₙᵢ—.

40. Compounds and the enantiomers, diastereoisomers and pharmaceutically acceptable salts thereof according to claim 3 wherein the compounds are:

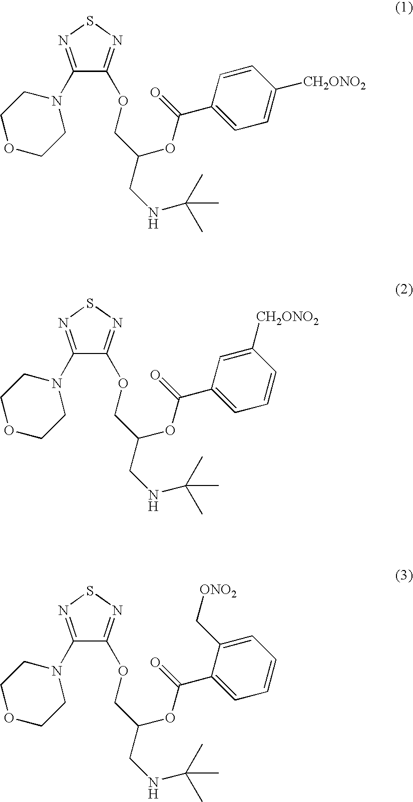

41. Compounds and the enantiomers, diastereoisomers and pharmaceutically acceptable salts thereof according to claim 20 wherein the compounds are:

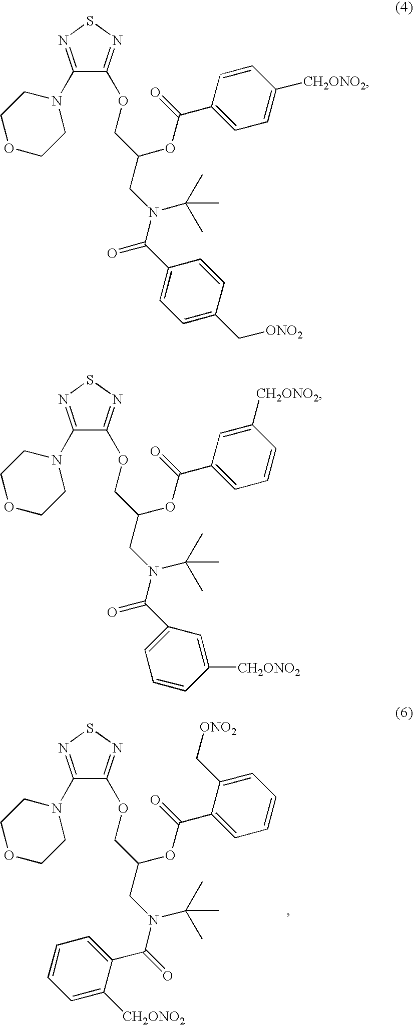

42. Compounds and the enantiomers, diastereoisomers and pharmaceutically acceptable salts thereof according to claim 30 wherein the compounds are:

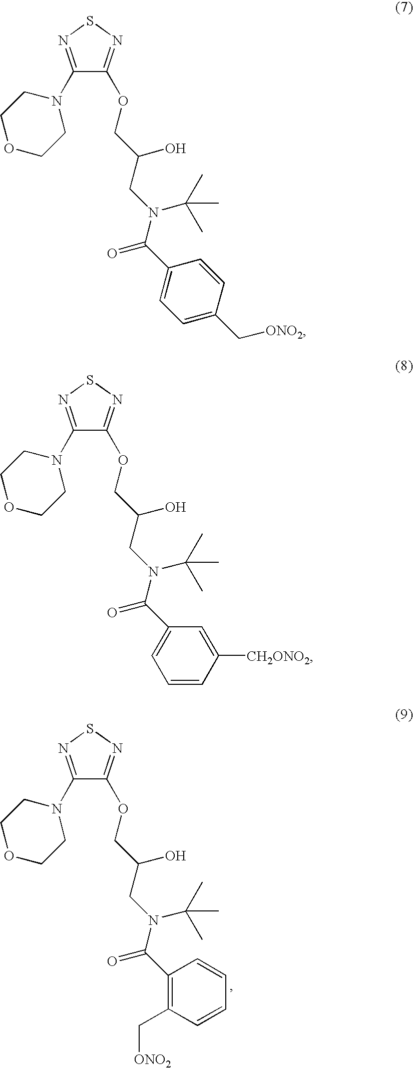

43. A compound and the enantiomers, diastereoisomers and pharmaceutically acceptable salts according to claim 1 which is 4-(Nitrooxymethyl)benzoic acid (S)-1-[(1,1-dimethylethyl)amino]-3-[[4-(4-morpholinyl)-1,2,5-thiadiazol-3-yl]oxy]-2-propanoate maleate salt.

44. A compound and the enantiomers, diastereoisomers and pharmaceutically acceptable salts according to claim 1, which is 4-(Nitrooxymethyl)benzoic acid (S)-1-[(1,1-dimethylethyl)[(4-nitrooxymethyl)benzoyl]amino]-3-[[4-(4-morpholinyl)-1,2,5-thiadiazol-3-yl]oxy]-2-propanoate.

45. A compound and the enantiomers, diastereoisomers and pharmaceutically acceptable salts according to claim 1 which is (S)-1-[(1,1-dimethylethyl)[(4-nitrooxymethyl)benzoyl]amino]-3-[[4-(4-morpholinyl)-1,2,5-thiadiazol-3-yl]oxy]-2-propanol.

46. A compound of formula (I) and/or the enantiomers, diastereoisomers and pharmaceutically acceptable salts thereof as defined in claim 1, for use as medicament.

47. Use of a compound of formula (I) and/or the enantiomers, diastereoisomers and pharmaceutically acceptable salts thereof as defined in claim 1 for preparing a drug that can be employed in the treatment or prophylaxis of hypertension, cardiovascular and vascular diseases.

48. Use of a compound of formula (I) and/or the enantiomers, diastereoisomers and pharmaceutically acceptable salts thereof as defined in claim 1 for preparing a drug that can be employed in the treatment of glaucoma and of elevated intraocular pressure.

49. A pharmaceutical composition comprising a compound of formula (I) and/or the enantiomers, diastereoisomers and pharmaceutically acceptable salts thereof as defined in claim 1 and a pharmaceutical acceptable carrier.
